SEQUENCE LISTING (30 sequences)

SEQ ID NO: 1 - DNA, 20 bp, Mus sp.
tccatgacgttcctgacgtt

SEQ ID NO: 2 - DNA, 16 bp, Homo sapiens
taacgttgaggggcat

SEQ ID NO: 3 - DNA, 16 bp, Homo sapiens
taagcatacggggtgt

SEQ ID NO: 4 - DNA, 16 bp, Homo sapiens
taacgttgaggggcat

SEQ ID NO: 5 - DNA, 6 bp, Homo sapiens
aacgtt

SEQ ID NO: 6 - DNA, 24 bp, Homo sapiens
gatgctgtgtcggggtctccgggc

SEQ ID NO: 7 - DNA, 24 bp, Homo sapiens
tcgtcgttttgtcgttttgtcgtt

SEQ ID NO: 8 - DNA, 20 bp, Homo sapiens
tccaggacttctctcaggtt

SEQ ID NO: 9 - DNA, 18 bp, Homo sapiens
tctcccagcgtgcgccat

SEQ ID NO: 10 - DNA, 20 bp, Mus sp.
tgcatcccccaggccaccat

SEQ ID NO: 11 - DNA, 20 bp, Homo sapiens
gcccaagctggcatccgtca

SEQ ID NO: 12 - DNA, 20 bp, Homo sapiens
gcccaagctggcatccgtca

SEQ ID NO: 13 - DNA, 15 bp, Homo sapiens
ggtgctcactgcggc

SEQ ID NO: 14 - DNA, 16 bp, Homo sapiens
aaccgttgaggggcat

SEQ ID NO: 15 - DNA, 24 bp, Homo sapiens
tatgctgtgccggggtcttcgggc

SEQ ID NO: 16 - DNA, 18 bp, Homo sapiens
gtgccggggtcttcgggc

SEQ ID NO: 17 - DNA, 18 bp, Homo sapiens
ggaccctcctccggagcc

SEQ ID NO: 18 - DNA, 18 bp, Homo sapiens
tcctccggagccagactt

SEQ ID NO: 19 - DNA, 15 bp, Homo sapiens
aacgttgaggggcat

SEQ ID NO: 20 - DNA, 15 bp, Homo sapiens
ccgtggtcatgctcc

SEQ ID NO: 21 - DNA, 21 bp, Homo sapiens
cagcctggctcaccgccttgg

SEQ ID NO: 22 - DNA, 20 bp, Mus sp.
cagccatggttccccccaac

SEQ ID NO: 23 - DNA, 20 bp, Homo sapiens
gttctcgctggtgagtttca

SEQ ID NO: 24 - DNA, 18 bp, Homo sapiens
tctcccagcgtgcgccat

SEQ ID NO: 25 - DNA, 15 bp, Homo sapiens
gtgctccattgatgc

SEQ ID NO: 26 - RNA, 33 nt, Homo sapiens
gaguucugaugaggccgaaaggccgaaagucug

SEQ ID NO: 27 - DNA, 6 bp, Artificial sequence
  synthetic
rrcgyy

SEQ ID NO: 28 - DNA, 15 bp, Homo sapiens
aacgttgaggggcat

SEQ ID NO: 29 - DNA, 16 bp, Homo sapiens
caacgttatggggaga

SEQ ID NO: 30 - DNA, 16 bp, Homo sapiens
taacgttgaggggcat

(19) United States
(12) Patent Application Publication
(10) Pub. No.: US 2003/0125292 A1
(43) Pub. Date: Jul. 3, 2003

(54) Mucoscal vaccine and methods for using the same

(76) Inventors: Sean Semple, Vancouver (CA); Sandy Klimuk, Vancouver (CA); Zuan-Ning Yuan, Vancouver (CA)

Correspondence Address: Todd A. Lorenz, DORSEY & WHITNEY LLP, Suite 3400, Four Embarcadero Center, San Francisco, CA, 94111-4187, US

(21) Appl. No.: 10/290,545

(22) Filed: Nov. 7, 2002

Related U.S. Application Data
(60) Provisional application No. 60/337,522, filed on Nov. 7, 2001.
(60) Provisional application No. 60/379,343, filed on May 10, 2002.

(51) Int. Cl. A61K 48/00; A61K 9/127; C12N 15/88

(52) U.S. Cl. 514/44; 424/450; 435/458

(57) ABSTRACT

The present invention relates to compositions and methods for stimulating enhanced mucosal immune responses in vivo. Particularly, the present invention relates to lipid-nucleic acids (“LNA”) formulations and methods of using thereof for stimulating enhanced mucosal immune responses in mammals. More particularly, the present invention relates to improved mucosal vaccines comprising target antigens associated with LNA formulations and methods of using thereof that stimulate antigen-specific mucosal immune responses in mammals.

### RELATED APPLICATIONS

This application claims priority to U.S. Provisional Application Serial No. 60/337,522, filed Nov. 7, 2001, and to U.S. Provisional Application Serial No. 60/379,343, filed May 10, 2002, under 35 U.S.C. §119(e).

### FIELD OF THE INVENTION

The present invention provides methods and compositions for stimulating enhanced mucosal immune responses in mammals. In particular, the present invention provides improved mucosal vaccines comprising immunostimulatory lipid-nucleic acid formulations in association with target antigens of interest, and methods of using such compositions.

### BACKGROUND OF THE INVENTION

The immune system broadly comprises the systemic immune system including bone marrow, spleen, and lymph nodes; and the mucosal immune system including lymphoid tissue associated with external secretory glands and mucosal surfaces (see, e.g., Staats et al., Curr. Opin. Immunol. 6:572-583 (1994)). The primary sites of transmission of most infectious diseases are the mucosal surfaces. Thus, the development of vaccines that can induce or enhance mucosal immunity is highly desirable (for review article see, e.g., McCluskie et al., Microbes and Infection 1:685-698 (1999)).

Due to the protective barriers of mucosal surfaces, traditional vaccines have been largely ineffective unless co-administered with specific mucosal adjuvants. In addition, many traditional mucosal vaccines are composed of live attenuated pathogens which carry the risk of reversion to virulent forms, particularly in immunocompromised individuals. Further, vaccines based on attenuated pathogens are limited because many pathogens cannot be attenuated.

Recombinant and synthetic antigens are considered safer than traditional vaccines composed of attenuated or inactivated microorganisms. However, the recombinant and synthetic antigens are often weakly immunogenic and therefore also necessitate the co-administration of adjuvants to enhance or induce specific antigenic immunity. The most common adjuvants used in animal models are cholera toxin (“CT”) and E. coli heat-labile enterotoxin (“LT”), which are toxic to humans (see, e.g., Wu and Russell, Infect Immun., 61:314-322 (1993); Staats et al., J. Immunol., 1:462-472 (1996); Gallichan and Rosenthal, Vaccine, 13:1589-1595 (1995); and Kuklin et al., J. Virol., 21:3138-3145 (1997)).

The potential of DNA vaccines to effectively induce systemic immune responses has been demonstrated in many species, including humans (Donelly et al., Annu. Rev. Immunol. 617-648, 15(1997); Davis et al., Microbes Infect. 7-23, 1(1999)). However, the majority of DNA vaccines have been delivered parenterally (e.g., via intramuscular or intradermal administration) and do not induce mucosal immune responses. Thus, systemic immunization that can provide systemic immunity may not provide mucosal immunity and, consequently, would not protect against mucosal infection (Lehner et al., Nature Med., 2:767-775 (1996)).

DNA vaccines which have been administered to an animal systemically or mucosally include adenovirus constructs that express reporter proteins and viral antigens. However, these constructs induce CD8+ T cells reactive to both the reporter protein and viral antigens of the adenoviral construct which causes clearance of adenovirus-infected cells from the animal within 10-14 days following administration (Yang et al., J. Virol., 69:2004-2015 (1995); Yang et al., Gene Therapy, 3:137-144 (1995)). These adenoviral recombinant constructs also stimulate CD4+ T helper cells (primarily the Th1 type) which promote activation of an antibody response and, thereby, prevents efficient re-infection of a second administration of the adenoviral vaccine. Thus, the strong immune response to the adenovirus vaccine itself diminishes the needed secondary immune response to the antigen expressed by the recombinant vaccine following administration of the booster.

In order to address the limitation of adenoviral recombinant vaccines, genetic vaccines based on plasmid vectors have been tested for their ability to induce a protective immune response in animals. Some studies demonstrated that upon systemic administration, plasmid-based vaccines prime the systemic immune system for a second systemic immunization with a traditional antigen, such as a protein or a recombinant virus (Xiang et al, Springer Semin. Immunopathol., 19:257-268 (1997); J. Schneider et al, Nature Med., 4:397 (1998); M. Sedeguh et al., Proc. Natl. Acad. Sci., U.S.A; 95:7648 (1998)). However, only low levels of genital IgA secretion were stimulated using plasmid-based vaccines co-administered with CT (Kuklin et al., J. Virol., 71:3138-3145 (1997)). Therefore, plasmid-based vaccines, which are useful for inducing a systemic immune response, may not be adequate for inducing a protective mucosal immune response.

Since the mid-1980's it has been known that nucleic acids, like other macromolecules, can act as biological response modifiers and induce immune responses in mammals upon in vivo administration (Tokunaga et al., 1984; Shimada et al., 1985; Mashiba et al, 1988; Yamamoto et al., 1988; Phipps et al. 1988). In the early 1990's it was established that stimulation of an immune response may be dependent on the features of the nucleic acid employed, for example, secondary structure palindromes (Yamamoto 1992a); methylation status of C nucleotides—depending on bacterial or mammalian source of DNA (Messina et al. 1991; Yamamoto 1992a); internucleotide linkage chemistry, e.g., phosphorothioates (Pisetsky and Reich 1993)); and specific nucleotide sequences, e.g., poly dG and CpG dinucleotide motifs (Tokunaga et al. 1992; Yamamoto et al 1992b; McIntyre, K W et al. 1993; Pisetsky and Reich, 1993; Yamamoto et al. 1994; Krieg et al. 1995). Such nucleic acid sequences that stimulate immune responses are called immune stimulatory sequences (“ISS”).

Attempts have been made to combine nucleic acids having an ISS with reduced amounts of CT to form a mucosal adjuvant (see, e.g., McCluskie and Davis, J. Immunol. (1998) 161(9):4463-4466. However, even with the reduced amounts of CT, such adjuvants still have associated toxicities and side effects that make them impractical for use as pharmacological agents. Moreover, the delivery of nucleic acids or other therapeutic agents to mucosal surfaces (e.g., genitourinary, gastrointestinal, and respiratory tracts) has been problematic due to enzymatic degradation and inefficient uptake of these components. For example, free nucleic acids are typically modified to incorporate a phophorothioate (“PS”) backbone in order to make them less susceptible to degradation. However, such PS modification can impede, or in some cases completely eliminate, the immunostimulatory activity of the free nucleic acids (see, e.g., Hartmann and Krieg, J. Immunol. (2000) 164:944-952. Thus, there is a need for formulating immunostimulatory compositions, e.g. nucleic acids, for more efficient delivery by increasing uptake and limiting the degradation of these compositions.

In view of the above, there is a great need for new and improved immunostimulatory compositions and methods that are capable of stimulating potent mucosal and systemic immune responses without associated toxicities. Further, there is a need for improved vaccine formulations comprising nucleic acids or other therapeutic agents that are protected from degradation and efficiently delivered to mucosal surfaces, in vivo. Accordingly, an object of the present invention is to provide safe and efficacious immunostimulatory compositions, and methods for using such compositions, for stimulating enhanced antigen-specific mucosal immune responses in mammals.

### SUMMARY OF THE INVENTION

In accordance with the above objects, the present invention provides compositions and methods for stimulating enhanced mucosal immune responses in mammals. The present invention is based on the discovery that combinations of nucleic acids and lipids can act synergistically to stimulate enhanced mucosal immune responses in vivo, as compared to the free or unencapsulated form of the nucleic acids. The present invention is further based on the discovery that such lipid-nucleic acid (“LNA”) formulations associated with a target antigen stimulate enhanced mucosal immune responses directed to that target antigen in vivo, as compared to the target antigen alone or mixed with the free or unencapsulated form of the nucleic acids.

In one embodiment, the LNA formulations of the present invention comprise a lipid component comprising a mixture of lipids, and a nucleic acid component comprising at least one oligonucleotide, preferably an oligodeoxynucleotide (“ODN”). In one aspect, reduced amounts of nucleic acids or other therapeutic agents can be used in the compositions of the present invention to stimulate enhanced mucosal immune responses, as compared to the free or unencapsulated form of the nucleic acids or other therapeutic agents. In another aspect, higher amounts of nucleic acids or other therapeutic agents can be used in comparison with the prior art to further enhance the response.

In a preferred embodiment, the invention provides a method for stimulating an enhanced mucosal immune response in a mammal comprising administering to the mammal an effective amount of an immunostimulatory composition comprising an LNA formulation in combination with at least one antigen, where the LNA formulation comprises: a) a lipid component comprising at least one lipid; and b) a nucleic acid component comprising at least one oligonucleotide, wherein the immunostimulatory composition stimulates an increased production of IgA as compared to the free form of the oligonucleotide, in vivo. In a particularly preferred embodiment, the LNA formulation is associated with the at least one antigen.

In a further embodiment, an improved method of stimulating production of IgA in mucosal tissues in a mammal is provided, comprising the administration to the mammal of an LNA formulation according to the present invention. Preferably the LNA formulation is administered in combination with at least one antigen of interest, and more preferably, the LNA formulation is associated with the antigen or antigens of interest. In one aspect, the administering is by intranasal delivery. In another aspect, the administering is by intradermal or subcutaneous delivery. In an additional aspect, the administering is by ex vivo delivery.

In another preferred embodiment, the invention provides an improved mucosal adjuvant comprising an LNA formulation, where the LNA formulation comprises: a) a lipid component comprising at least one lipid; and b) a nucleic acid component comprising at least one oligonucleotide, wherein the nucleic acid component is encapsulated by the lipid component, and the lipid component and the nucleic acid component act synergistically to stimulate immunoglobulin A (IgA) production in a mammal. In one aspect, the subject LNA formulations are capable of eliciting an IgA response that is at least one-fold, more preferably at least two-fold, and most preferably three- or four-fold higher than that obtained using the free nucleic acid utilized in the prior art.

In a further preferred embodiment, the invention provides an improved mucosal vaccine composition comprising an LNA formulation associated with at least one antigen, where the LNA formulation comprises: a) a lipid component comprising at least one lipid; and b) a nucleic acid component comprising at least one oligonucleotide, wherein the nucleic acid component is encapsulated by the lipid component, and the lipid component and said nucleic acid component act synergistically to stimulate antigen-specific IgA production in a mammal. In a particularly preferred embodiment, the at least one antigen is attached to or encapsulated by the LNA formulation. In one aspect, the antigen-specific IgA production obtained using the improved mucosal vaccine compositions described herein is at least one or two-fold greater than that achieved by administering either free nucleic acid or an LNA formulation mixed with the antigen, and more preferably at least three- or four-fold greater.

In one embodiment, the lipid component of the LNA formulation comprises a cationic lipid. In a further embodiment, the cationic lipid is selected from a group of cationic lipids consisting of DDAB, DODAC, DOTAP, DMRIE, DOSPA, DMDMA, DC-Chol, DOGS, DODMA, and DODAP.

In a further embodiment, the lipid component of the LNA formulation comprises a neutral lipid. In a further embodiment, the neutral lipid is selected from a group of neutral lipids consisting of DOPE, DSPC, POPC, diacylphosphatidylcholine, diacylphosphatidylethanolamine, ceramide, sphingomyelin, cephalin, and cerebrosides.

In preferred embodiments, the lipid component of the LNA formulation comprises DSPC, DODMA, Chol, and PEG-DMG and the ratio of the DSPC to the DODMA to the Chol to the PEG-DMG is about 20:25:45:10 mol/mol. In one aspect, the ratio of the lipid component to the nucleic component of the LNA formulations of the compositions and methods of the present invention is about 0.01-0.25 wt/wt. In another aspect, the lipid component of the LNA formulations of the compositions and methods of the present invention comprises a lipid membrane encapsulating said oligonucleotide.

In one embodiment, the nucleic acid component of the LNA formulation comprises at least one oligonucleotide that is an oligodeoxynucleotide (ODN). In a preferred embodiment, the ODN comprises at least one CpG dinucleotide. In one aspect, the CpG dinucleotide is methylated or unmethylated. In a particularly preferred embodiment, the ODN is selected from a group of ODNs consisting of ODN #1, ODN #2, ODN #3, ODN #4, ODN #5, ODN #6, ODN #7, ODN #8, and ODN #9. In an additional aspect, the ODN comprises a phosphorothioate backbone (ODN PS).

In an additional aspect, the LNA formulations of the compositions and methods of the present invention further comprise an antigen. In an additional aspect the antigen is attached to the LNA. In an additional aspect, the lipid component of the LNA formulations of the compositions and methods of the present invention comprise a lipid membrane having an external portion and an internal portion, and the antigen is attached to said external portion of said lipid membrane.

### BRIEF DESCRIPTION OF THE DRAWINGS

FIG. 1 depicts the titer of anti-OVA IgG (FIG. 1A), anti-OVA IgA (FIG. 1B), and anti-OVA IgM (FIG. 1C) in serum on day 28 following the initial immunization of C57BL/6 mice (6 weeks old) with 20 μl of the test formulations listed below in the order depicted (from left to right) by intranasal administration on day 0 (initial immunization), and days 7, and 14 after the initial immunization. The mice received OVA protein at a dose of 75 μg per immunization, and the free or encapsulated ODN were administered at doses of 1, 10 and 100 μg.

OVA alone

OVA co-administered with 10 μg CT (“OVA+CT”)

OVA co-administered with ODN #1 (“OVA+ODN #1”)

OVA co-administered with ODN #2 (“OVA+ODN #2”)

OVA co-administered with ODN #3 (“OVA+ODN #3”)

OVA co-administered with LNA containing ODN #1 (“OVA+LNA-ODN #1”)

OVA co-administered with LNA containing ODN #2 (“OVA+LNA-ODN #2”)

OVA co-administered with LNA containing ODN #3 (“OVA+LNA-ODN #3”)

LNA containing ODN #2 (“LNA-ODN #2”)

FIG. 2 depicts the titer of anti-OVA IgG (FIG. 2A), anti-OVA IgA (FIG. 2B), and anti-OVA IgM (FIG. 2C) in lung washes on day 28 following the initial immunization of C57BL/6 mice (6 weeks old) with 20 μl of the test formulations listed below in the order depicted (from left to right) by intranasal administration on day 0 (initial immunization), and days 7, and 14 after the initial immunization. The mice received OVA protein at a dose of 75 μg per immunization, and the free or encapsulated ODN were administered at doses of 1, 10 and 100 μg.

OVA alone

OVA co-administered with 10 μg CT (“OVA+CT”)

OVA co-administered with ODN #1 (“OVA+ODN #1”)

OVA co-administered with ODN #2 (“OVA+ODN #2”)

OVA co-administered with ODN #3 (“OVA+ODN #3”)

OVA co-administered with LNA containing ODN #1 (“OVA+LNA-ODN #1”)

OVA co-administered with LNA containing ODN #2 (“OVA+LNA-ODN #2”)

OVA co-administered* with LNA containing ODN #3 (“OVA+LNA-ODN #3”)

LNA containing ODN #2 (“LNA-ODN #2”)

FIG. 3 depicts the titer of anti-OVA IgG (FIG. 3A) and anti-OVA IgA (FIG. 3B) in vaginal washes on day 28 following the initial immunization of C57BL/6 mice (6 weeks old) with 20 μl of the test formulations listed below in the order depicted (from left to right) by intranasal administration on day 0 (initial immunization), and days 7, and 14 after the initial immunization. The mice received OVA protein at a dose of 75 μg per immunization, and the free or encapsulated ODN were administered at doses of 1, 10 and 100 μg.

OVA alone

OVA co-administered with 10 μg CT (“OVA+CT”)

OVA co-administered with ODN #1 (“OVA+ODN #1”)

OVA co-administered with ODN #2 (“OVA+ODN #2”)

OVA co-administered with ODN #3 (“OVA+ODN #3”)

OVA co-administered with LNA containing ODN #1 (“OVA+LNA-ODN #1”)

OVA co-administered with LNA containing ODN #2 (“OVA+LNA-ODN #2”)

OVA co-administered* with LNA containing ODN #3 (“OVA+LNA-ODN #3”)

LNA containing ODN #2 (“LNA-ODN #2”)

FIG. 4 depicts humoral immunity as indicated by the titer of anti-OVA IgG in serum (FIG. 4A), lung wash (FIG. 4B), and vaginal wash (FIG. 4C) on day 28 following the initial immunization of C57BL/6 mice (6 weeks old) with 20 μl of the test formulations listed below in the order depicted (from left to right) by intranasal administration on day 0 (intial immunization), and days 7, and 14 after the initial immunization. The mice received OVA protein at a dose of 75 μg per immunization, and the free or encapsulated ODN were administered at doses of 10 and 100 μg.

OVA co-administered with ODN #2 PS (“OVA+ODN #2 PS”) at a dose of 10 μg

OVA co-administered with ODN #2 PS (“OVA+ODN #2 PS”) at a dose of 100 μg

OVA co-administered with LNA containing ODN #2 PS (“OVA+LNA-ODN #2 PS”) at a dose of 10 μg

OVA co-administered with LNA containing ODN #2 PS (“OVA+LNA #2 PS”) at a dose of 100 μg

OVA coupled to LNA containing ODN #2 (“OVA/LNA-ODN #2 PS”) at a dose of 10 μg

OVA coupled to LNA containing ODN #2 (“OVA/LNA-ODN #2 PS”) at a dose of 100 μg

OVA co-administered with 10 μg of CT (“OVA+CT”)

OVA co-administered with LNA containing ODN #1 (“OVA/LNA-ODN #1 PS”) at a dose of 10 μg

FIG. 5 depicts humoral immunity as indicated by the titer of anti-OVA IgA in serum (FIG. 5A), lung wash (FIG. 5B), and vaginal wash (FIG. 5C) on day 28 following the initial immunization of C57BL/6 mice (6 weeks old) with 20 μl of the test formulations listed below in the order depicted (from left to right) by intranasal administration on day 0 (intial immunization), and days 7, and 14 after the initial immunization. The mice received OVA protein at a dose of 75 μg per immunization, and the free or encapsulated ODN were administered at doses of 10 and 100 μg.

OVA co-administered with ODN #2 PS (“OVA+ODN #2 PS”) at a dose of 10 μg

OVA co-administered with ODN #2 PS (“OVA+ODN #2 PS”) at a dose of 100 μg

OVA co-administered with LNA containing ODN #2 PS (“OVA+LNA-ODN #2 PS”) at a dose of 10 μg

OVA co-administered with LNA containing ODN #2 PS (“OVA+LNA #2 PS”) at a dose of 100 μg

OVA coupled to LNA containing ODN #2 (“OVA/LNA-ODN #2 PS”) at a dose of 10 μg

OVA coupled to LNA containing ODN #2 (“OVA/LNA-ODN #2 PS”) at a dose of 100 μg

OVA co-administered CT (“OVA+CT”) at a dose of 10 μg

OVA co-administered with LNA containing ODN #1 (“OVA/LNA-ODN #1 PS”) at a dose of 10 μg

FIG. 6 depicts the titer of anti-OVA IgA in lung washes was on day 28 following the initial immunization of C57BL/6 mice (6 weeks old) with 20 μl of the test formulations listed below in the order depicted (from left to right) by intranasal administration on day 0 (intial immunization), and days 7, and 14 after the initial immunization. The mice received OVA protein at a dose of 75 μg per immunization, and the free or encapsulated ODN were administered at doses of 100 μg.

PBS alone

OVA co-administered with 10 μg CT (“OVA+CT”)

LNA containing ODN #2 (“LNA-ODN #2”)

OVA co-administered with ODN #1 (“OVA+ODN #1”)

OVA co-administered with LNA containing ODN #1 (“OVA+LNA-ODN #1”)

OVA co-administered with ODN #2 (“OVA+ODN #2”)

OVA co-administered with LNA containing ODN #2 (“OVA+LNA-ODN #2”)

OVA co-administered with ODN #3 (“OVA+ODN #3”)

OVA co-administered* with LNA containing ODN #3 (“OVA+LNA-ODN #3”)

FIG. 7 depicts the titer of anti-OVA IgA in vaginal washes was on day 28 following the initial immunization of C57BL/6 mice (6 weeks old) with 20 μl of the test formulations listed below in the order depicted (from left to right) by intranasal administration on day 0 (intial immunization), and days 7, and 14 after the initial immunization. The mice received OVA protein at a dose of 75 μg per immunization, and the free or encapsulated ODN were administered at doses of 100 μg.

PBS alone

OVA co-administered with 10 μg CT (“OVA+CT”)

LNA containing ODN #2 (“LNA-ODN #2”)

OVA co-administered with ODN #1 (“OVA+ODN #1”)

OVA co-administered with LNA containing ODN #1 (“OVA+LNA-ODN #1”)

OVA co-administered with ODN #2 (“OVA+ODN #2”)

OVA co-administered with LNA containing ODN #2 (“OVA+LNA-ODN #2”)

OVA co-administered with ODN #3 (“OVA+ODN #3”)

OVA co-administered* with LNA containing ODN #3 (“OVA+LNA-ODN #3”)

FIG. 8 depicts the titer of anti-OVA IgA in lung washes (FIG. 8A) and vaginal washes (FIG. 8B) on day 28 following the initial immunization of C57BL/6 mice (6 weeks old) with 20 μl of the test formulations listed below in the order depicted (from left to right) by intranasal administration on day 0 (intial immunization), and days 7, and 14 after the initial immunization. The mice received OVA protein at a dose of 75 μg per immunization, and the free or encapsulated ODN were administered at doses of 10 and 100 μg.

OVA coupled to LNA containing ODN #2 (“OVA/LNA-ODN #2 PS”) at a dose of 100 μg

OVA coupled to LNA containing ODN #2 (“OVA/LNA-ODN #2 PS”) at a dose of 10 μg

OVA co-administered with LNA containing ODN #2 PS (“OVA+LNA #2 PS”) at a dose of 100 μg

OVA co-administered with LNA containing ODN #2 PS (“OVA+LNA #2 PS”) at a dose of 10 μg

OVA co-administered with 100 μg of ODN #2 PS (“OVA+ODN #2 PS”)

OVA co-administered with 10 μg of ODN #2 PS (“OVA+ODN #2 PS”)

OVA co-administered with 10 μg CT (“OVA+CT”) PBS alone

FIG. 9 depicts the titer of anti-OVA IgG in lung washes (FIG. 9A) and vaginal washes (FIG. 9B) on day 28 following the initial immunization of C57BL/6 mice (6 weeks old) with 20 μl of the test formulations listed below in the order depicted (from left to right) by intranasal administration on day 0 (intial immunization), and days 7, and 14 after the initial immunization. The mice received OVA protein at a dose of 75 μg per immunization, and the free or encapsulated ODN were administered at doses of 10 and 100 μg.

OVA coupled to LNA containing ODN #2 (“OVA/LNA-ODN #2 PS”) at a dose of 100 μg

OVA coupled to LNA containing ODN #2 (“OVA/LNA-ODN #2 PS”) at a dose of 10 μg

OVA co-administered with LNA containing ODN #2 PS (“OVA+LNA #2 PS”) at a dose of 100 μg

OVA co-administered with LNA containing ODN #2 PS (“OVA+LNA #2 PS”) at a dose of 10 μg

OVA co-administered with ODN #2 PS (“OVA+ODN #2 PS”) at a dose of 100 μg

OVA co-administered with ODN #2 PS (“OVA+ODN #2 PS”) at a dose of 10 μg

OVA co-administered with 10 μg CT (“OVA+CT”)

PBS alone

FIG. 10 depicts the titer of anti-OVA IgG in plasma on day following the initial immunization of C57BL/6 mice (6 weeks old) with 20 μl of the test formulations listed below in the order depicted (from left to right) by intranasal administration on day 0 (intial immunization), and days 7, and 14 after the initial immunization. The mice received OVA protein at a dose of 75 μg per immunization, and the free or encapsulated ODN were administered at doses of 10 and 100 μg.

OVA coupled to LNA containing ODN #2 (“OVA/LNA-ODN #2 PS”) at a dose of 100 μg

OVA coupled to LNA containing ODN #2 (“OVA/LNA-ODN #2 PS”) at a dose of 10 μg

OVA co-administered with LNA containing ODN #2 PS (“OVA+LNA #2 PS”) at a dose of 100 μg

OVA co-administered with LNA containing ODN #2 PS (“OVA+LNA #2 PS”) at a dose of 10 μg

OVA co-administered with ODN #2 PS (“OVA+ODN #2 PS”) at a dose of 100 μg

OVA co-administered with ODN #2 PS (“OVA+ODN #2 PS”) at a dose of 10 μg

OVA co-administered with 10 μg CT (“OVA+CT”)

PBS alone

### DETAILED DESCRIPTION

The present invention provides compositions and methods for stimulating enhanced mucosal immune responses in mammals. In particular, the present invention provides compositions comprising nucleic acids and lipids that act synergistically to stimulate enhanced mucosal immune responses in vivo, as compared to the free or unencapsulated form of the nucleic acids. Further, these lipid-nucleic acids (“LNA”) formulations can be associated with target antigens to stimulate potent mucosal responses directed to the target antigens, in vivo. Moreover, enhanced mucosal immune responses may be stimulated using reduced amounts of nucleic acids or other therapeutic agents in the immunostimulatory LNA formulations of the present invention, as compared to known immunostimulatory compositions. Alternatively, using the compositions and methods of the present invention, higher amounts of nucleic acids or other therapeutic agents, may be administered as compared to known immunostimulatory compositions.

A hallmark of an effective mucosal adjuvant or vaccine is the ability of the adjuvant to stimulate production of immunoglobulin A (“IgA”) antibodies which neutralize pathogens in or adjacent to mucosal epithelial cells (see, e.g., Lamm et al, Vaccine Res. (1992) 1:169). Activated IgA cell precursors can migrate to other mucosal sites and differentiate into plasma cells that secrete IgA (secretory IgA or S-IgA) (see, e.g., McGhee et al. Vaccine (1992) 10:75). Thus, the potent production of antigen-specific IgA antibodies at sites local and distal to the site of immunization is desirable for an effective and lasting mucosal immune response.

The present invention is based on the discovery that combinations of nucleic acids and lipids can act synergistically to stimulate enhanced mucosal immune responses in vivo, resulting in significantly increased IgA titers as compared to the free or unencapsulated form of the nucleic acids. Thus, reduced amounts of nucleic acids may be used in the LNA formulations of the present invention to stimulate enhanced mucosal immune responses in vivo, as compared to the free or unencapsulated form of the nucleic acids. Moreover, higher concentrations of the LNA formulations of the present invention may be administered as compared to known immunostimulatory compositions comprising free nucleic acids, because in such known immunostimulatory compositions, the free nucleic acids can exhibit toxicity at elevated concentrations or exhibit a plateau in the dose response curve with increasing concentration of the free nucleic acids.

Additionally, the present invention is based on the discovery that a significant improvement in antigen-specific IgA production may be obtained by administering a target antigen of interest in combination with the LNA formulations of the present invention. In a preferred embodiment, methods are provided for stimulating enhanced antigen-specific mucosal immune responses, using vaccine compositions comprising LNA formulations in association with target antigens of interest.

The mucosal vaccine compositions of the present invention provide a significant advantage in that antigen and adjuvant can be simultaneously delivered via the liposomal particles directly to immune cells of interest, e.g., macrophages. Significant stimulation of mucosal immune responses to the target antigen, including enhancements in the nature of the responses, can be realized as compared to the weak immunogenic responses rendered by some immunogens alone, or by the simple mixing of adjuvants and immunogens disclosed in the prior art. See, e.g., PCT publication WO 98/40100; U.S. Pat. No. 6,406,705; McCluskie and Davis (1998); Gallichan et al., J. Immunol. 3451-3457 (2001). Thus, the vaccine compositions of the present invention provide a more potent mucosal vaccine as compared to traditional or known vaccines.

The LNA formulations described herein provide additional advantages over known immunostimulatory compositions. For example, as compared to formulations of free nucleic acids, the LNA formulations of the present invention stimulate significantly improved mucosal immune responses in vivo. Further, LNA formulations comprising ODNs having a phosphorothioate backbone (ODN PS) can be used in the methods of the present invention to stimulate an enhanced immune response in vivo, as compared to the free form phosphodiester oligonucleotides. Moreover, free nucleic acids that are not effectively immunostimulatory, or are non-immunostimulatory, provide an immunostimulatory effect when formulated in the LNA formulations of the present invention.

In additional and alternative embodiments, the methods of the present invention use LNA formulations comprising antisense nucleic acids that stimulate synergistic immune responses and targeted antisense activity. Also, the co-administration of LNA formulations and cytotoxic agents (e.g., doxorubicin) in the methods of the present invention stimulate synergistic immune responses and targeted cytoxic activity.

Abbreviations and Definitions

The following abbreviations are used herein: RBC, red blood cells; DDAB, N,N-distearyl-N,N-dimethylammonium bromide; DODAC, N,N-dioleyl-N,N-dimethylammonium chloride; DOPE, 1,2-sn-dioleoylphoshatidylethanolamine; DOSPA, 2,3-dioleyloxy-N-(2(sperminecarboxamido)ethyl)-N,N-dimethyl-1-propanaminiu m trifluoroacetate; DOTAP, 1,2-dioleoyloxy-3-(N,N,N-trimethylamino)propane chloride; DOTMA, 1,2-dioleyloxy-3-(N,N,N-trimethylamino)propanechloride; OSDAC, N-oleyl-N-stearyl-N,N-dimethylammonium chloride; RT, room temperature; HEPES, 4-(2-hydroxyethyl)-1-piperazineethanesulfonic acid; FBS, fetal bovine serum; DMEM, Dulbecco's modified Eagle's medium; PEG-Cer-C.sub.14, 1-O-(2′-(.omega.-methoxypolyethyleneglycol)succinoyl)-2-N-myristoyl-sphing osine; PEG-Cer-C.sub.20, 1-O-(2′-(.omega.-methoxypolyethyleneglycol)succinoyl)-2-N-arachidoyl-sphin gosine; PBS, phosphate-buffered saline; THF, tetrahydrofuran; EGTA, ethylenebis(oxyethylenenitrilo)-tetraacetic acid; SF-DMEM, serum-free DMEM; and NP40, nonylphenoxypolyethoxyethanol.

The technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entirety as though set forth in full. Standard reference works setting forth the general principles of recombinant DNA technology include Sambrook, J., et al., Molecular Cloning,: A Laboratory Manual, 2d Ed., Cold Spring Harbor Laboratory Press, Planview, N.Y. (1989); McPherson, M. J., Ed., Directed Mutagenesis: A Practical Approach, IRL Press, Oxford (1991); Jones, J., Amino Acid and Peptide Synthesis, Oxford Science Publications, Oxford (1992); Austen, B. M. and Westwood, O. M. R., Protein Targeting and Secretion, IRL Press, Oxford (1991). Any suitable materials and/or methods known to those of skill can be utilized in carrying out the present invention; however, preferred materials and/or methods are described. Materials, reagents and the like to which reference is made in the following description and examples are obtainable from commercial sources, unless otherwise noted. It is believed that one skilled in the art can, based on the description herein, utilize the present invention to its fullest extent. The entire contents of all of the references (including literature references, issued patents, published patent applications, and co-pending patent applications) cited throughout this application are hereby expressly incorporated by reference.

The immunostimulatory compositions used in the methods of the present invention will generally be lipid-therapeutic agent (“LTA”) formulations comprising at least one lipid component and at least one therapeutic agent, and having greater immunostimulatory activity than the therapeutic agent alone, in vivo. “Therapeutic agent” or “therapeutic compound” or “drug” as used herein can be used interchangeably and refer to any synthetic, recombinant, or naturally occurring molecule that provides a beneficial effect in medical treatment of a subject. Examples of therapeutic agents include, but are not limited to, nucleic acids, peptides, and chemicals.

In the preferred embodiments described herein, the therapeutic agent comprises at least one nucleic acid, more preferably at least one oligonucleotide, and most preferably at least one oligodeoxynucleotide (“ODN”) in an LNA formulation. In a particularly preferred embodiment, the ODN comprises an immunostimulatory sequence (“ISS”). “ISS” as used herein refers to nucleic acid sequences that can can stimulate immune responses in mammals upon in vivo administration (Tokunaga et al., 1984; Shimada et al., 1985; Mashiba et al., 1988; Yamamoto et al., 1988; Phipps et al. 1988). In a preferred embodiment, the ISS comprises a CpG motif (Tokunaga et al. 1992; Yamamoto et al 1992b; McIntyre, K W et al. 1993; Pisetsky and Reich, 1993; Yamamoto et al. 1994; Krieg et al. 1995). In another preferred embodiment, the ODN comprises at least one CpG motif. Methylated and unmethylated CpG motifs are both useful in the compositions and methods of the present invention.

“Subject” as used herein refers to an organism, male or female, having an immune system, preferably an animal, more preferably a vertebrate, even more preferably a mammal, still even more preferably a rodent, and most preferably a human. Further examples of a subject include, but are not limited to, dogs, cats, cows, horses, pigs, sheep, goats, mice, rabbits, and rats. “Patient” as used herein refers to a subject in need of treatment for a medical condition (e.g., disease or disorder).

“In vivo” as used herein refers to an organism, preferably in a mammal, more preferably in a rodent, and most preferably in a human.

“Immunostimulatory” or “stimulating an immune response,” or grammatical equivalents thereof, as used herein refers to inducing, increasing, enhancing, or modulating an immune response, or otherwise providing a beneficial effect with respect to an immune response. As used herein “immune response” refers to systemic and/or mucosal immune response

By “mucosal immune response” or “mucosal immunity” as the terms are interchangeably used herein, is meant the induction of a humoral (i.e., B cell) and/or cellular (i.e., T cell) response and may be assessed using methods well known in the art. For example, a humoral mucosal immune response may be assessed by measuring the antigen-specific antibodies present in the mucosal lavage in response to the introduction of the desired antigen into the host. Also for example, the mucosal immune response may be assessed by measuring antigen-specific antibody titers and isotype profiles in vaginal lavage of immunized mammals. In a preferred embodiment, the antibody response (of a mucosal immune response) is comprised primarily of immunoglobulin A (“IgA”) antibodies, and more preferably secreted IgA (“S-IgA”). Also for example, a cellular mucosal immune response may be assessed by measuring the T cell response from lymphocytes isolated from a mucosal area (e.g., vagina or gastrointestinal tract) or from lymph nodes that drain from a mucosal area (e.g., genital area or gastrointestinal area). The invention should be construed to include the immune response of the various mucosa of mammals of either gender and of various species.

The enhanced mucosal immune response obtained according to the present invention may be demonstrated and determined in a variety of ways, including, for example, the production of enhanced levels of cytokine and/or immunoglobulin in mucosal tissues. Also for example, the levels of immunostimulatory activity of the compositions and methods of the present invention may compared to the level of immunostimulatory activity of known adjuvants and vaccines. In preferred embodiments, the immunostimulatory activity of the LNA formulations of the present invention comprising an antigen may be compared to the immunostimulatory activity of the nucleic acid component alone (e.g., free nucleic acids), the nucleic acid component mixed with the antigen, LNA mixed with the antigen, or the antigen alone.

In a preferred embodiment, the LNA compositions and methods of the present invention stimulate the production of immunoglobulin A (“IgA”) titers that are at least two fold, and more preferably at least three fold higher as compared to the nucleic acids alone. In another preferred embodiment, the vaccine compositions and methods of the present invention stimulate the productions of antigen-specific IgA titers that are at least two-fold higher, and more preferably three fold higher, as compared to known mucosal vaccines.

“Target antigen” as used herein refers to an antigen of interest to which a immune response can be directed or stimulated. The target antigen used in the compositions of the present invention for stimulating an immune response directed to that target antigen may be a synthetic, naturally-occuring or isolated molecule or a fragment thereof, and may comprise single or multiple epitopes. Thus, the compositions of the present invention may stimulate immune responses directed to single or multiple epitopes of an antigen. In preferred embodiments, the target antigen is associated with the LNA formulations of the present invention. “In association with, “associated with”, or grammatical equivalents thereof, as used herein with reference to an antigen (or target antigens), refers to antigens that are attached to or encapsulated by another component. With reference to the lipid particles or liposomes of the present invention, the antigen may be, for example, encapsulated in the lumen or intralamellar spaces of the lipid particles; disposed or attached within or partially within the lipid membrane, or attached (e.g., covalently or ionically) to the lipid particle. The antigen may be attached to the interior of the lipid particle or, more preferably, the antigen is attached to the exterior of the lipid particle.

Examples of antigens useful in the compositions and methods of the present invention include, but are not limited to, peptides or proteins, cells, cell extracts, polysaccharides, polysaccharide conjugates, lipids, glycolipids, glycopeptides, and carbohydrates. In one embodiment, the antigen is in the form of a peptide or protein antigen. In another embodiment, the antigen is a nucleic acid encoding a peptide or protein in a form suitable for expression in a subject and presentation to the immune system of that subject. In a preferred embodiment, the compositions used in the methods of the present invention comprise a peptide or protein target antigen that stimulates an immune response to that target antigen in a mammal. Preferably, the target antigen is a pathogen (“target pathogen”) capable of infecting a mammal including, for example, bacteria, viruses, fungi, yeast, parasites and other microorganisms capable of infecting mammalian species.

The term “antigen” is further intended to encompass peptide or protein analogs of known or wild-type antigens such as those described above. The analogs may be more soluble or more stable than wild type antigen, and may also contain mutations or modifications rendering the antigen more immunologically active. Also useful in the compositions and methods of the present invention are peptides or proteins which have amino acid sequences homologous with a desired antigen's amino acid sequence, where the homologous antigen induces an immune response to the respective pathogen.

“Homologous” as used herein refers to the subunit sequence similarity between two polymeric molecules, e.g., between two nucleic acid molecules (e.g., two DNA molecules or two RNA molecules) or two polypeptide molecules. When a subunit position in both molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g. five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-CCGTTA-3′ and 5′-GCGTAT-3′ share 50% homology. By the term “substantially homologous” as used herein, is meant DNA or RNA which is about 50% homologous, more preferably about 70% homologous, even more preferably about 80% homologous and most preferably about 90% homologous to the desired nucleic acid. Genes which are homologous to the desired antigen-encoding sequence should be construed to be included in the invention provided they encode a protein or polypeptide having a biological activity substantially similar to that of the desired antigen. Where in this text, protein and/or DNA sequences are defined by their percent homologies or identities to identified sequences, the algorithms used to calculate the percent homologies or percent identities include the following: the Smith-Waterman algorithm (J. F. Collins et al, Comput. Appl. Biosci., (1988) 4:67-72; J. F. Collins et al, Molecular Sequence Comparison and Alignment, (M. J. Bishop et al, eds.) In Practical Approach Series: Nucleic Acid and Protein Sequence Analysis XVIII, IRL Press: Oxford, England, UK (1987) 417), and the BLAST and FASTA programs (E. G. Shpaer et al, 1996, Genomics, 38:179-191). These references are incorporated herein by reference.

Analogs of the antigens described herein can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Modifications (which do not normally alter primary sequence) include in vivo, or in vitro chemical derivatization of polypeptides, e.g., acetylation, or carboxylation. Also contemplated as antigens are proteins modified by glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also contemplated as antigens are amino acid sequences which have phosphorylated amino acid residues, e.g., phosphotyrosine, phosphoserine, or phosphothreonine. Also contemplated as antigens are polypeptides which have been modified using ordinary molecular biological techniques so as to improve their resistance to proteolytic degradation or to optimize solubility properties. Analogs of such polypeptides include those containing residues other than naturally occurring L-amino acids, e.g., D-amino acids or non-naturally occurring synthetic amino acids.

The antigens of the present invention are not limited to products of any of the specific exemplary processes listed herein. In addition to substantially full length polypeptides, the antigens useful in the present invention include immunologically active fragments of the polypeptides. For example, the antigen may be a fragment of a complete antigen including at least one therapeutically relevant epitope. “Therapeutically relevant epitope” as used herein refers to an epitope for which the mounting of an immune response in a subject against the epitope will provide a therapeutic benefit for that subject. In preferred embodiments, a fragment (of a complete antigen) which may be highly immunogenic, but which does not produce an immune response directed to the complete antigen or antigenic source (e.g., a microorganism) would not be a “therapeutically relevant epitope.” Also useful in the compositions and methods of the present invention are combination antigens which include multiple epitopes from the same target antigen, or epitopes from two or more different target antigents (i.e., polytope vaccines). For example, the combination antigens can be the same or different type such as, e.g., a peptide-peptide antigen, glycolipid-peptide antigen, or glycolipid-glycolipid antigen.

A polypeptide or antigen is “immunologically active” if it induces an immune response to a target antigen or pathogen. “Vaccine” as used herein refers to a composition comprising a target antigen that stimulates a specific immune response to that target antigen.

“Adjuvant” as used herein refers to any substance which can stimulate immune responses, preferably mucosal immune responses. Some adjuvants can cause activation of a cell of the immune system, for example, an adjuvant can cause an immune cell to produce and secrete cytokines. Examples of adjuvants that can cause activation of a cell of the immune system include, but are not limited to, saponins purified from the bark of the Q. saponaria tree, such as QS21 (a glycolipid that elutes in the 21^{st} peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”). As compared to known adjuvants, the present invention provides improved adjuvants comprising combinations of lipids and nucleic acids that act synergistically to stimulate enhanced immune responses. In preferred embodiments, such LNA formulations of the present invention comprise a nucleic acid component and a lipid component Preferrably the nucleic acid component comprises at least one oligonucleotide, more preferably at least one ODN, and most preferably at least one ODN comprising at least one CpG motif.

In preferred embodiments the immunostimulatory compositions used in the methods of the present invention comprise a lipid component comprising a lipid membrane that encapsulates a therapeutic agent. As used herein “liposome,” “lipid vesicle,” and “liposomal vesicle,” or grammatical equivalents thereof, may be used interchangeably and refer to structures, particles, complexes, or formulations comprising lipid-containing membranes which enclose or encapsulate an aqueous interior. In preferred embodiments, the liposomes enclose or encapsulate therapeutic agents, e.g., nucleic acids. The liposomes may have one or more lipid membranes. In preferred embodiments, the liposomes have one membrane. Liposomes having one lipid-containing membrane are referred to herein as “unilamellar.” Liposomes having multiple lipid-containing membranes are referred to herein as “multilamellar.” “Lipid bilayer” as used herein refers to a lipid-containing membrane having two layers.

Mucosal Adjuvants Comprising Lipid-Nucleic Acid Formulations

The immunostimulatory compositions used in the methods of the present invention generally comprise an LNA formulation, which can be used either alone as an adjuvant or associated with target antigen in a vaccine composition. As noted above, the LNA formulation will typically comprise at least one lipid component and at least one nucleic acid component.

Nucleic Acids

Nucleic acids suitable for use in the LNA formulations of the present invention include, for example, DNA or RNA. Preferably the nucleic acids are oligonucleotides, more preferably ODNs, and more preferably ODN comprising an ISS (“ISS ODN”).

“Nucleic acids” as used herein refer to multiple nucleotides (i.e., molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)). Nucleic acids may be, for example DNA or RNA. Preferably the nucleic acids are oligoribonucleotides and more preferably ODNs. Nucleic acids may also be polynucleosides, i.e., a polynucleotide minus the phosphate and any other organic base containing polymer. In preferred embodiments, the LNA formulations of the present invention comprise a nucleic acid component. “Nucleic acid component” as used herein with reference to the LNA formulations of the present invention refer to a component comprising nucleic acids.

In a preferred embodiment, the oligonucleotides are single stranded and in the range of 6-50 nucleotides (“nt”) in length. However, any oligonucleotides may be used including, for example, large double stranded plasmid DNA in the range of 500-50,000 base pairs (“bp”).

Nucleic acids useful in the compositions and methods of the present invention can be obtained from known sources or isolated using methods well known in the art. The nucleic acids can also be prepared by recombinant or synthetic methods which are equally well known in the art. Such nucleic acids can then be prepared in LNA formulations and the resulting compositions tested for immunostimulatory activity using the methods of the present invention as described herein.

Oligonucleotides useful in the compositions and methods of the present invention may have a modified backbone, although as indicated above such modification is not required as is the case in the prior art. Modified oligonucleotides include phosphodiester modified oligonucleotide, combinations of phosphodiester (“PO”) and phosphorothioate (“PS”) oligonucleotide, methylphosphonate, methylphosphorothioate, phosphorodithioate, and combinations thereof. In addition, other modified oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated.

Numerous other chemical modifications to the base, sugar or linkage moieties are also useful. Bases may be methylated or unmethylated. Nucleotide sequences useful in the compositions and methods of the present invention may be complementary to patient/subject mRNA, such as antisense oligonucleotides, or they may be foreign or non-complementary (e.g., the nucleotide sequences do not specifically hybridize to the patient/subject genome). The nucleotide sequences may be expressed and the resulting expression products may be RNA and/or protein. In addition, such nucleotide sequences may be linked to appropriate promoters and expression elements, and be contained in an expression vector. Nucleotide sequences useful in the composition and methods of the present invention may be ISS, such as certain palindromes leading to hairpin secondary structures (see Yamamoto S., et al. (1992) J. Immunol. 148: 4072-4076), or CpG motifs, or other known ISS features (such as multi-G domains, see WO 96/11266).

The nucleic acids of the present invention can be synthesized de novo using any of a number of procedures well known in the art. For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986,; Garegg et al., Tet. Let. 27:4055-4058, 1986, Gaffney et al., Tet. Let. 29:2619-2622, 1988). These chemistries can be performed by a variety of automated oligonucleotide synthesizers available in the market. Also, CpG dinucleotides can be produced on a large scale in plasmids, (see Sambrook, T., et al., Molecular Cloning: A Laboratory Manual, Cold Spring Harbor laboratory Press, New York, 1989). Such plasmids may also encode other genes to be expressed such as an antigen-encoding gene in the case of a DNA vaccine. Oligonucleotides can be prepared from existing nucleic acid sequences (e.g., genomic or cDNA) using known techniques, such as those employing restriction enzymes, exonucleases or endonucleases.

For use in vivo, nucleic acids are preferably relatively resistant to degradation (e.g., via endo- and exo-nucleases). Secondary structures, such as stem loops, can stabilize nucleic acids against degradation. Alternatively, nucleic acid stabilization can be accomplished via phosphate backbone modifications. A preferred stabilized nucleic acid has at least a partial phosphorothioate modified backbone. Phosphorothioates may be synthesized using automated techniques employing either phosphoramidate or H-phosphonate chemistries. Aryl-and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990).

For administration in vivo, compositions of the present invention, including components of the compositions, e.g., a lipid component or a nucleic acid component, may be associated with a molecule that results in higher affinity binding to target cell (e.g., B-cell, monocytic cell and natural killer (NK) cell) surfaces and/or increased cellular uptake by target cells. The compositions of the present invention, including components of the compositions, can be ionically or covalently associated with desired molecules using techniques which are well known in the art. A variety of coupling or cross-linking agents can be used, e.g., protein A, carbodiimide, and N-succinimidyl-3-(2-pyridyidithio) propionate (SPDP).

The immune stimulating activity of a nucleic acid sequence in an organism can be determined by simple experimentation, for example, by comparing the sequence in question with other immunostimulatory agents, e.g., other adjuvants, or ISS; or by detecting or measuring the immunostimulatory activity of the sequence in question, e.g., by detecting or measuring the activation of host defense mechanisms or the activation of immune system components. Such assays are well known in the art. Also, one of skill in the art would know how to identify the optimal oligonucleotides useful for a particular mammalian species of interest using routine assays described herein and/or known in the art.

The nucleic acid sequences of ODNs suitable for use in the compositions and methods of the invention are described in U.S. Patent Appln. 60/379,343, U.S. Patent Appln. No. 09/649,527, Int. Publ. WO 02/069369, Int. Publ. No. WO 01/15726, U.S. Pat. No. 6,406,705, and Raney et al., Journal of Pharmacology and Experimental Therapeutics, 298:1185-1192 (2001), which are all incorporated herein by reference. Exemplary sequences of the ODNs include, but are not limited to, those nucleic acid sequences shown in Table 1. In preferred embodiments, ODNs used in the compositions and methods of the present invention have a phosphodiester (“PO”) backbone or a phosphorothioate (“PS”) backbone.

TABLE 1

| | | |
|---|---|---|
| ODN NAME | ODN SEQ ID NO | ODN SEQUENCE (5′-3′) |

| | | |
|---|---|---|
| ODN #1 (INX-1826) | SEQ ID NO:1 | 5′-TCCATGACGTTCCTGACGTT-3 |
| ODN #2 (INX-6295) | SEQ ID NO:2 | 5′-TAACGTTGAGGGGCAT-3 |
| human c-myc | | |
| ODN #3 (INX-6300) | SEQ ID NO:3 | 5′-TAAGCATACGGGGTGT-3 |
| ODN #4 (INX-6303) | SEQ ID NO:4 | 5′-TAACGTTGAGGGGCAT-3 |
| ODN #5 (INX-5001) | SEQ ID NO:5 | 5′-AACGTT-3 |
| ODN #6 (INX-3002) | SEQ ID NO:6 | 5′-GATGCTGTGTCGGGGTCTCCGGGC3′ |
| ODN #7 (INX-2006) | SEQ ID NO:7 | 5′-TCGTCGTTTTGTCGTTTTGTCGTT3′ |
| ODN #8 (INX-1982) | SEQ ID NO:8 | 5′-TCCAGGACTTCTCTCAGGTT-3′ |
| ODN #9 (INX-G3139) | SEQ ID NO:9 | 5′-TCTCCCAGCGTGCGCCAT-3′ |
| ODN #10 (PS-3082) | SEQ ID NO:10 | 5′-TGCATCCCCCAGGCCACCAT3 |
| murine Intracellular | | |
| Adhesion Molecule-1 | | |
| ODN #11 (PS-2302) | SEQ ID NO:11 | 5′-GCCCAAGCTGGCATCCGTCA-3′ |
| human Intracellular | | |
| Adhesion Molecule-1 | | |
| ODN #12 (PS-8997) | SEQ ID NO:12 | 5′-GCCCAAGCTGGCATCCGTCA-3′ |
| human Intracellular | | |
| Adhesion Molecule-1 | | |
| ODN #13 (US3) | SEQ ID NO:13 | 5′-GGT GCTCACTGC GGC-3′ |
| human erb-B-2 | | |
| ODN #14 (LR-3280) | SEQ ID NO:14 | 5′-AACC GTT GAG GGG CAT-3′ |
| human c-myc | | |
| ODN #15 (LR-3001) | SEQ ID NO:15 | 5′-TAT GCT GTG CCG GGG TCT TCG GGC-3′ |
| human c-myc | | |
| ODN #16 (lnx-6298) | SEQ ID NO:16 | 5′-GTGCCG GGGTCTTCGGGC-3′ |
| ODN #17 (hIGF-1R) | SEQ ID NO:17 | 5′-GGACCCTCCTCCGGAGCC-3′ |
| human Insulin Growth | | |
| Factor 1 - Receptor | | |
| ODN #18 (LR-52) | SEQ ID NO:18 | 5′-TCG TCC GGA 0CC AGA CTT-3′ |
| human Insulin Growth | | |
| Factor 1 - Receptor | | |
| ODN #19 (hEGFR) | SEQ ID NO:19 | 5′-AAC GTT GAG GGG CAT-3′ |
| human Epidermal | | |
| Growth Factor - | | |
| Receptor | | |
| ODN #20 (EGFR) | SEQ ID NO:20 | 5′-CCGTGGTCA TGCTCC-3′ |
| Epidermal Growth | | |
| Factor - Receptor | | |
| ODN #21 (hVEGF) | SEQ ID NO:21 | 5′-CAG CCTGGCTCACCG CCTTGG-3′ |
| human Vascular | | |
| Endothelial Growth | | |
| Factor | | |
| ODN #22 (PS-4189) | SEQ ID NO:22 | 5′-CAG CCATGG TTC CCC CCAAC-3′ |
| murine Phosphokinase | | |
| C - alpha | | |
| ODN #23 (PS-3521) | SEQ ID NO:23 | 5′-GTT CTC GCT GGT GAG TTT CA-3′ |
| ODN #24 (hBcl-2) | SEQ ID NO:24 | 5′-TCT CCCAGCGTGCGCCAT-3′ |
| human Bcl-2 | | |
| ODN #25 (hC-Raf-1) | SEQ ID NO:25 | 5′-GTG CTC CAT TGA TGC-3′ |
| human C-Raf-s | | |
| ODN #26 (hVEGF-R1) SEQ ID NO:26 | 5′-GAGUUCUGAUGAGGCCGAAAGGCCG | |
| | | AAAGUCUG-3′ |
| human Vascular | | |
| Endothelial Growth | | |
| Factor Receptor-1 | | |
| ODN #27 | SEQ ID NO:27 | 5′-RRCGYY-3′ |
| ODN #28 (INX-3280) | SEQ ID NO:28 | 5′-AACGTTGAGGGGCAT-3′ |
| ODN #29 (INX-6302) | SEQ ID NO:29 | 5′-CAACGTTATGGGGAGA-3′ |
| ODN #30 (INX-6298) | SEQ ID NO:30 | 5′-TAACGTTGAGGGGCAT-3′ |
| human c-myc | | |

Lipids and Other Components

Lipid formulations and methods of preparing liposomes as delivery vehicles are known in the art. Preferred lipid formulations are described herein and more fully described in, for example, U.S. Pat. No. 5,785,992, U.S. Pat. No. 6,287,591, U.S. Pat. No. 6,287,591 B1, co-pending U.S. Patent Appln. Ser. No. 60/379,343, and co-pending U.S. application Ser. No. 09/649,527, all incorporated herein by reference.

In one preferred embodiment, the preferred lipid formulation is DSPC, DODMA, Chol, and PEG-DMG having a ratio of 20:25:45:10 mol/mol. As used herein, the molar amount of each lipid in a lipid formulation is given in the same order that the lipid is listed (e.g., the ratio of DSPC to DODMA to Chol to PEG-DMG is 20 DSPC: 25 DODMA: 45 Chol; 10 PEG-DMG or “20:25:45: 10”).

The term “lipid” refers to a group of organic compounds that are esters of fatty acids and are characterized by being insoluble in water but soluble in many organic solvents. They are usually divided in at least three classes: (1) “simple lipids” which include fats and oils as well as waxes; (2) “compound lipids” which include phospholipids and glycolipids; and (3) “derived lipids” such as steroids and compounds derived from lipid manipulations. A wide variety of lipids may be used with the invention, some of which are described below.

The term “charged lipid” refers to a lipid species having either a cationic charge or negative charge or which is a zwitterion which is not net neutrally charged, and generally requires reference to the pH of the solution in which the lipid is found.

Cationic charged lipids at physiological pH include, but are not limited to, N,N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-dioleyloxy)propyl)-N,N,N-trimethylammonium chloride (“DOTAP”); 3b-(N-(N′,N′-dimethylaminoethane)-carbamoyl)cholesterol (“DC-Chol”) and N-(1,2-dimyristyloxyprop-3-yl)-N,N-dimethyl-N-hydroxyethyl ammonium bromide (“DMRIE”). Additionally, a number of commercial preparations of catioinic lipids are available which can be used in the present invention. These include, for example, Lipofectin™ (commercially available cationic liposomes comprising DOTMA and 1,2-dioleoyl-sn-3-phosphoethanolamine (“DOPE”), from GIBCO/BRL, Grand Island, N.Y., U.S.A); Lipofectamine™ (commercially available cationic liposomes comprising N-(1-(2,3-dioleyloxy)propyl)-N-(2-(sperminecarboxamido)ethyl)-N,N-dimethylammonium trifluoroacetate (“DOSPA”) and DOPE from GIBCO/BRL); and Transfectam™ (commercially available cationic lipids comprising dioctadecylamidoglycyl carboxyspermine (“DOGS”) in ethanol from Promega Corp., Madison, Wis., U.S.A).

Some cationic charged lipids are titratable, that is to say they have a pKa at or near physiological pH, with the significant consequence for this invention that they are strongly cationic in mild acid conditions and weakly (or not) cationic at physiological pH. Such cationic charged lipids include, but are not limited to, N-(2,3-dioleyloxy)propyl)-N,N-dimethylammonium chloride (“DODMA”) and 1,2-Dioleoyl-3-dimethylammonium-propane (“DODAP”). DMDMA is also a useful titratable cationic lipid.

Anionic charged lipids at physiological pH include, but are not limited to, phosphatidyl inositol, phosphatidyl serine, phosphatidyl glycerol, phosphatidic acid, diphosphatidyl glycerol, poly(ethylene glycol)-phosphatidyl ethanolamine, dimyristoylphosphatidyl glycerol, dioleoylphosphatidyl glycerol, dilauryloylphosphatidyl glycerol, dipalmitoylphosphatidyl glycerol, distearyloylphosphatidyl glycerol, dimyristoyl phosphatic acid, dipalmitoyl phosphatic acid, dimyristoyl phosphatidyl serine, dipalmitoyl phosphatidyl serine, brain phosphatidyl serine, and the like.

Some anionic charged lipids may be titrateable, that is to say they would have a pKa at or near physiological pH, with the significant consequence for this invention that they are strongly anionic in mild base conditions and weakly (or not) anionic at physiological pH. Such anionic charged lipids can be identified by one skilled in the art based on the principles disclosed herein.

The term “neutral lipid” refers to any of a number of lipid species which exist either in an uncharged or neutral zwitterionic form at physiological pH. Such lipids include, for example, diacylphosphatidylcholine, diacylphosphatidylethanolamine, ceramide, sphingomyelin, cephalin, cholesterol, cerebrosides and diacylglycerols.

Certain preferred lipid formulations used in the invention include aggregation preventing compounds such as PEG-lipids or polyamide oligomer-lipids (such as an ATTA-lipid), and other steric-barrier or “stealth”-lipids, detergents, and the like. Such lipids are described in U.S. Pat. No. 4,320,121, U.S. Pat. No. 5,820,873, U.S. Pat. No. 5,885,613, Int. Publ. No. WO 98/51278, and U.S. patent application Ser. No. 09/218,988 relating to polyamide oligomers, all incorporated herein by reference. These lipids and detergent compounds prevent precipitation and aggregation of formulations containing oppositely charged lipids and therapeutic agents. These lipids may also be employed to improve circulation lifetime in vivo (see Klibanov et al. (1990) FEBS Letters, 268 (1): 235-237), or they may be selected to rapidly exchange out of the formulation in vivo (see U.S. Pat. No. 5,885,613, incorporated herein by reference).

A preferred embodiment of the invention employs exchangeable steric-barrier lipids (as described in U.S. Pat. No. 5,820,873, U.S. Pat. No. 5,885,613, and U.S. patent application Ser. No. 09/094,540 and U.S. Pat. No. 6,320,017, all assigned to the assignee of the present invention and all incorporated herein by reference). Exchangeable steric-barrier lipids such as PEG2000-CerC14 and ATTA8-CerC14 are steric-barrier lipids which rapidly exchange out of the outer monolayer of a lipid particle upon administration to a subject/patient. Each such lipid has a characteristic rate at which it will exchange out of a particle depending on a variety of factors including acyl chain length, saturation, size of steric barrier moiety, membrane composition and serum composition, etc. Such lipids are useful in preventing aggregation during particle formation, and their accelerated departure from the particle upon administration provides benefits, such as programmable fusogenicity and particle destabilizing activity, as described in the above noted patent submissions.

Some lipid particle formulations may employ targeting moieties designed to encourage localization of liposomes at certain target cells or target tissues. Targeting moieties may be associated with the outer bilayer of the lipid particle (i.e., by direct conjugation, hydrophobic interaction or otherwise) during formulation or post-formulation. These methods are well known in the art. In addition, some lipid particle formulations may employ fusogenic polymers such as PEM, hemagluttinin, other lipo-peptides (see U.S. Pat. No. 6,417,326, and U.S. patent application Ser. No. 09/674,191, all incorporated herein by reference) and other features useful for in vivo and/or intracellular delivery.

In another preferred embodiment, the lipid component of the LNA formulations of the present invention comprises sphingomyelin and cholesterol (“sphingosomes”). In a preferred embodiment, the LNA formulations used in the compositions and methods of the present invention are comprised of sphingomyelin and cholesterol and have an acidic intraliposomal pH. The LNA formulations comprising sphingomyelin and cholesterol have several advantages when compared to other formulations. The sphingomyelin/cholesterol combination produces liposomes which have extended circulation lifetimes, are much more stable to acid hydrolysis, have significantly better drug retention characteristics, have better loading characteristics into tumors and the like, and show significantly better anti-tumor efficacy than other liposomal formulations tested.

In a preferred embodiment, the ratio of sphingomyelin to cholesterol is in the range of about 75/25 mol %/mol sphingomyelin/cholesterol to 30/50 mol %/mol % sphingomyelin/cholesterol, more preferably about 70/30 mol %/mol sphingomyelin/cholesterol to 40/45 mol %/mol % sphingomyelin/cholesterol, and most preferably is approximately 55/45 mol %/mol % sphingomyelin/cholesterol. Other lipids may be present in the formulations as may be necessary, for example, to prevent lipid oxidation or to attach ligands onto the liposome surface.

In a preferred embodiment, the LNA formulations of the present invention comprise a cationic compound of Formula I and at least one neutral lipid as follows (and fully described in U.S. Pat. No. 5,785,992, incorporated herein by reference).

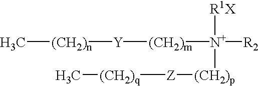

In Formula I, R¹and R² are each independently C₁ to C₃; alkyl. Y and Z are akyl or alkenyl chains and are each independently: —CH₂CH₂CH₂CH₂CH₂—, —CH═CHCH₂CH₂CH₂—, —CH₂ CH═CHCH₂CH₂—, —CH₂CH₂CH═CHCH₂—, —CH₂CH₂CH₂CH═CH—, —CH═CHCH═CHCH₂—, —CH═CHCH₂CH═CH—, or —CH₂CH═CHCH═CH—, with the proviso that Y and Z are not both —CH₂CH₂CH₂CH₂CH₂—. The letters n and q denote integers of from 3 to 7, while the letters m and p denote integers of from 4 to 9, with the proviso that the sums n+m and q+p are each integers of from 10 to 14. The symbol X⁻ represents a pharmaceutically acceptable anion. In the above formula, the orientation of the double bond can be either cis or trans, however the cis isomers are generally preferred.

In another preferred embodiment, the cationic compounds are of Formula I, wherein R¹and R² are methyl and Y and Z are each independently: —CH═CHCH₂CH₂CH₂—, —CH₂CH═CHCH₂CH₂—, —CH₂CH₂CH═CHCH₂—or —CH₂CH₂CH₂CH═CH—. In preferred embodiments, R¹ and R² are methyl; Y and Z are each —CH═CHCH₂CH₂CH₂—; n and q are both 7; and m and p are both 5. In another preferred embodiment, the cationic compound is DODAC (N,N-dioleyl-N,N-dimethylammonium chloride). DODAC is a known in the art and is a compound used extensively as an additive in detergents and shampoos. DODA is also used as a co-lipid in liposomal compositions with other detergents (see, Takahashi, et al., GB 2147243).

The neutral lipids in the LNA formulations of the present invention can be any of a variety of neutral lipids which are typically used in detergents, or for the formation of micelles or liposomes. Examples of neutral lipids which are useful in the present compositions are, but are not limited to, diacylphosphatidylcholine, diacylphosphatidylethanolamine, ceramide, sphingomyelin, cephalin, cardiolipin, and cerebrosides. In a preferred embodiment, the present compositions will include one or more neutral lipids which are diacylphosphatidylcholine, diacylphosphatidylethanolamine, ceramide or sphingomyelin. The acyl groups in these neutral lipids are preferably acyl groups derived from fatty acids having C₁₀-C₂₄ carbon chains. More preferably the acyl groups are lauroyl, myristoyl, palmitoyl, stearoyl or oleoyl. In particularly preferred embodiments, the neutral lipid will be 1,2-sn-dioleoylphosphatidylethanolamine.

The anion, X³¹, can similarly be any of a variety a pharmaceutically acceptable anions. These anions can be organic or inorganic, including for example, Br, Cl⁻, F⁻, I⁻, sulfate, phosphate, acetate, nitrate, benzoate, citrate, glutamate, and lactate. In preferred embodiments, X⁻ is Cl⁻or AcO⁻.

In addition to the other components described herein, the compositions of the present invention may contain a pharmaceutically acceptable carrier. Pharmaceutically acceptable carriers are well-known in the art. The choice of carrier is determined in part by the particular composition to be administered as well as by the particular method used to administer the composition. Preferably, the pharmaceutical carrier is in solution, in water or saline.

In the compositions of the present invention, the ratio of cationic compound to neutral lipid is preferably within a range of from about 25:75 (cationic compound:neutral lipid), or preferably to 75:25 (cationic compound:neutral lipid), or preferably about 50:50.

The cationic compounds which are used in the compositions of the present invention can be prepared by methods known to those of skill in the art using standard synthetic reactions (see March, Advanced Organic Chemistry, 4th Ed., Wiley-Interscience, NY, N.Y. (1992), incorporated herein by reference). For example, the synthesis of OSDAC can be carried out by first treating oleylamine with formaldehyde and sodium cyanoborohydride under conditions which result in the reductive alklation of the amine. This approach provides dimethyl oleylamine, which can then be alkylated with stearyl bromide to form the corresponding ammonium salt. Anion exchange results in the formation of OSDAC. Dimethyloleylamine can also be synthesized by treatment of oleyl bromide with a large excess of dimethylamine, and further derivatized as described above.

For cationic compounds in which both fatty acid chains are unsaturated (i.e., DODAC), the following general procedure can be used. An unsaturated acid (i.e., oleic acid) can be converted to its corresponding acyl chloride with such reagents as oxalyl chloride, thionyl chloride, PCl₃ or PCl₅. The acyl chloride can be treated with an unsaturated amine (i.e., oleylamine) to provide the corresponding amide. Reduction of the amide with, for example, lithium aluminum hydride provides a secondary amine wherein both alkyl groups are unsaturated long chain alkyl groups. The secondary amine can then be treated with alkyl halides such as methyl iodide to provide a quaternary ammonium compound. Anion exchange can then be carried out to provide cationic compounds having the desired pharmaceutically acceptable anion. The alkylamine precursor can be synthesized in a similar manner. For example, treatment of an alkyl halide with a methanolic solution of ammonia in large excess will produce the required amine after purification. Alternatively, an acyl chloride, produced by treatment of the appropriate carboxylic acid with oxalyl chloride, can be reacted with ammonia to produce an amide. Reduction of the amide with LiAlH₄ will provide the required alkylamine.

In preferred embodiments, the pharmaceutical compositions of the present invention are formulated as micelles or liposomes. Micelles containing the cationic compounds and neutral lipids of the present invention can be prepared by methods well known in the art. In addition to the micellar formulations of the present compositions, the present invention also provides micellar formulations which include other species such as lysophosphatidylcholine, lysophosphatidylethanolamine, lysophosphatidylserine, lysophosphatidylglycerol, phosphatidylethanolamine-polyoxyethylene conjugate, ceramide-polyoxyethylene conjugate or phosphatidic acid-polyoxyethylene conjugate.

The polyoxyethylene conjugates which are used in the compositions of the present invention can be prepared by combining the conjugating group (i.e. phosphatidic acid or phosphatidylethanolamine) with an appropriately functionalized polyoxyethylene derivative. For example, phosphatidylethanolamine can be combined with omega-methoxypolyethyleneglycol succinate to provide a phosphatidylethanolamine-polyoxyethylene conjugate (see, e.g., Parr, et al., Biochim. Biophys. Acta 1195:21-30 (1994), incorporated herein by reference).

The selection of neutral lipids for use in the compositions and methods of the present invention is generally guided by consideration of, e.g., liposome size and stability of the liposomes in the bloodstream. As described above, the neutral lipid component in the liposomes is a lipid having two acyl groups, (i.e., diacylphosphatidylcholine and diacylphosphatidyl-ethanolamine). Lipids having a variety of acyl chain groups of varying chain length and degree of saturation are available or may be isolated or synthesized by well-known techniques. In general, less saturated lipids are more easily sized, particularly when the liposomes must be sized below about 0.3 microns, for purposes of filter sterilization. In one group of embodiments, lipids containing saturated fatty acids with carbon chain lengths in the range of C₁₄ to C₂₂ are preferred. In another group of embodiments, lipids with mono or diunsaturated fatty acids with carbon chain lengths in the range of C₁₄ to C₂₂ are used. Additionally, lipids having mixtures of saturated and unsaturated fatty acid chains can be used.

Liposomes useful in the compositions and methods of the present invention may also be composed of sphingomyelin or phospholipids with other head groups, such as serine and inositol. Still other liposomes useful in the present invention will include cholesterol, diglycerides, ceramides, phosphatidylethanolamine-polyoxyethylene conjugates, phosphatidic acid-polyoxyethylene conjugates, or polyethylene glycol-ceramide conjugates (e.g., PEG-Cer-C₁₄ or PEG-Cer-C₂₀). Methods used in sizing and filter-sterilizing liposomes are discussed below.

A variety of methods are known in the art for preparing liposomes (see e.g., Szoka et al., Ann. Rev. Biophys. Bioeng. 9:467 (1980), U.S. Pat. Nos. 4,235,871, 4,501,728, 4,837,028, the text Liposomes, Marc J. Ostro, ed., Marcel Dekker, Inc., New York, 1983, Chapter 1, and Hope, et al., Chem. Phys. Lip. 40:89 (1986), all of which are incorporated herein by reference). One known method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous buffered solution and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Following liposome preparation, the liposomes may be sized to achieve a desired size range and relatively narrow distribution of liposome sizes. A size range of about 0.2-0.4 microns allows the liposome suspension to be sterilized by filtration through a conventional filter, typically a 0.22 micron filter. The filter sterilization method can be carried out on a high through-put basis if the liposomes have been sized down to about 0.2-0.4 microns.

Several techniques are available for sizing liposomes to a desired size. One sizing method is described in U.S. Pat. No. 4,737,323, incorporated herein by reference. Sonicating a liposome suspension either by bath or probe sonication produces a progressive size reduction down to small unilamellar vesicles less than about 0.05 microns in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. In both methods, the particle size distribution can be monitored by conventional laser-beam particle size discrimination.

Extrusion of liposomes through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve a gradual reduction in liposome size. For use in the present inventions, liposomes having a size of from about 0.05 microns to about 0.15 microns are preferred.

As further described below, the compositions of the present invention can be administered to a subject by any known route of administration. Once adsorbed by cells, the liposomes (including the complexes previously described) can be endocytosed by a portion of the cells, exchange lipids with cell membranes, or fuse with the cells. Transfer or incorporation of the polyanionic portion of the complex can take place via any one of these pathways. In particular, when fusion takes place, the liposomal membrane can be integrated into the cell membrane and the contents of the liposome can combine with the intracellular fluid.

As described below in detail, additional components, which may also be therapeutic compounds, may be added to the LNA formulations of the present invention to target them to specific cell types. For example, the liposomes can be conjugated to monoclonal antibodies or binding fragments thereof that bind to epitopes present only on specific cell types, such as cancer-related antigens, providing a means for targeting the liposomes following systemic administration. Alternatively, ligands that bind surface receptors of the target cell types may also be bound to the liposomes. Other means for targeting liposomes may also be employed in the present invention.

Following a separation step as may be necessary to remove free drug from the medium containing the liposome, the liposome suspension is brought to a desired concentration in a pharmaceutically acceptable carrier for administration to the patie nt or host cells. Many pharmaceutically acceptable carriers may be employed in the compositions and methods of the present invention. A variety of aqueous carriers may be used, e.g., water, buffered water, 0.4% saline, 0.3% glycine, and the like, and may include glycoproteins for enhanced stability, such as albumin, lipoprotein, globulin. Generally, normal buffered saline (135-150 mM NaCl) will be employed as the pharmaceutically acceptable carrier, but other suitable carriers will suffice. These compositions may be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride. These compositions may be sterilized techniques referred to above or produced under sterile conditions. The resulting aqueous solutions may be packaged for use or filtered under aseptic conditions and lyophilized, the lyophilized preparation being combined with a sterile aqueous solution prior to administration.

The concentration of liposomes in the carrier may vary. In preferred embodiments, the concentration of liposomes is about 0.1-200 mg/ml. Persons of skill would know how to vary these concentrations to optimize treatment with different liposome components or for particular patients. For example, the concentration may be increased to lower the fluid load associated with treatment.

The cells of a subject are usually exposed to the LNA formulations of the present invention by in vivo or ex vivo administration. In the preferred embodiments described herein, the compositions of the present invention are administered intranasally or intratracheally. Intratracheal administration may be provided as a liquid, preferably as an aerosol. For example, nebulizers may be used to create aerosols of droplets of between 70-100 μm in diameter. It will be understood that droplet size should generally be of greater size than the liposomes.

Multiple administrations to a patient are contemplated. The dosage schedule of the treatments will be determined by the disease and the patient's condition. Standard treatments with therapeutic compounds, including immunostimulatory compositions (e.g., vaccines), that are well known in the art may serve as a guide to treatment with liposomes containing the therapeutic compounds. The duration and schedule of treatments may be varied by methods well known to those of skill, but the increased circulation time and decreased in liposome leakage will generally allow the dosages to be adjusted downward from those previously employed. The dose of liposomes of the present invention may vary depending on the clinical condition and size of the animal or patient receiving treatment. The standard dose of the therapeutic compound when not encapsulated may serve as a guide to the dose of the liposome-encapsulated compound. The dose will typically be constant over the course of treatment, although in some cases the dose may vary. Standard physiological parameters may be assessed during treatment that may be used to alter the dose of the liposomes of the invention.

Other Drug Components

Some preferred embodiments of the invention further comprise other therapeutic agents, e.g., drugs or bioactive agents. These additional components may provide direct additional therapeutic benefit or additional immune-stimulating benefits. A wide variety of therapeutic compounds may be delivered by the compositions and methods of the present invention. Examples of therapeutic compounds include, but are not limited to, nucleic acids, proteins, peptides, oncolytics, anti-infectives, anxiolytics, psychotropics, immunomodulators, ionotropes, toxins such as gelonin and inhibitors of eucaryotic protein synthesis, and the like. Preferred therapeutic compounds for entrapment in the liposomes of the present invention are those which are lipophilic cations. Among these are therapeutic agents of the class of lipophilic molecules which are able to partition into a lipid bilayer phase of a liposome, and which therefore are able to associate with the liposomes in a membrane form. Further examples of therapeutic compounds include, but are not limited to, prostaglandins, amphotericin B, methotrexate, cisplatin and derivatives, progesterone, testosterone, estradiol, doxorubicin, epirubicin, beclomethasone and esters, vitamin E, cortisone, dexamethasone and esters, betamethasone valerete and other steroids, the fluorinated quinolone antibacterial ciprofloxacin and its derivatives, and alkaloid compounds and their derivatives. Among the alkaloid derivatives are swainsonine and members of the vinca alkaloids and their semisynthetic derivatives, such as, e.g., vinblastine, vincristine, vindesin, etoposide, etoposide phosphate, and teniposide. Among this group, vinblastine and vincristine, and swainsonine are particularly preferred. Swainsonine (Creaven and Mihich, Semin. Oncol. 4:147 (1977) has the capacity to stimulate bone marrow proliferation (White and Olden, Cancer Commun. 3:83 (1991)). Swainsonine also stimulates the production of multiple cytokines including IL-1, IL-2, TNF, GM-CSF and interferons (Newton, Cancer Commun. 1:373 (1989); Olden, K., J. Natl. Cancer Inst., 83:1149 (1991)). Further Swainsonine reportedly induces B- and T-cell immunity, natural killer T-cell and macrophage-induced destruction of tumor cells in vitro, and when combined with interferon, has direct anti-tumor activity against colon cancer and melanoma cancers in vivo (Dennis, J., Cancer Res., 50:1867 (1990); Olden, K., Pharm. Ther. 44:85 (1989); White and Olden, Anticancer Res., 10:1515 (1990)). Other alkaloids useful in the compositions and methods of the present invention include, but are not limited to, paclitaxel (taxol) and synthetic derivatives thereof. Additional drug components, include but are not limited to, any bioactive agents known in the art which can be incorporated into lipid particles.

These additional drug components may, be encapsulated or otherwise associated with the LNA formulations described herein. Alternatively, the compositions of the invention may include drugs or bioactive agents that are not associated with the lipid-nucleic acid particle. Such drugs or bioactive agents may be in separate lipid carriers or co-administered.

Mucosal Vaccine Compositions

As described herein, the improved mucosal vaccine compositions of the present invention comprise the LNA formulations as described herein associated with at least one target antigen. Antigens useful in the compositions and methods of the present invention may be inherently immunogenic, or non-immunogenic, or slightly immunogenic. Examples of antigens include, but are not limited to, synthetic, recombinant, foreign, or homologous antigens. Further examples of antigens include, but are not limited to, HBA—hepatitis B antigen (recombinant or otherwise); other hepatitis peptides; HIV proteins GP120 and GP160; Mycoplasma cell wall lipids; any tumor associated antigen; Carcinoembryonic Antigen (CEA); other embryonic peptides expressed as tumor specific antigens; bacterial cell wall glycolipids; Gangliosides (GM2, GM3); Mycobacterium glycolipids; PGL-1; Ag85B; TBGL; Gonococcl lip-oligosaccharide epitope 2C7 from Neisseria gonorrhoeae; Lewis(y); and Globo-H; Tn; TF; STn; PorA; TspA or Viral glycolipids/glycoproteins and surface proteins.

The antigen may be in the form of a peptide antigen or it may be a nucleic acid encoding an antigenic peptide in a form suitable for expression in a subject and presentation to the immune system of the immunized subject. The antigen may also be a glycolipid or a glycopeptide. Further, the antigen may be a complete antigen, or it may be a fragment of a complete antigen including at least one therapeutically relevant epitope. “Combination antigens” as herein refer to antigens having multiple epitopes from the same target antigen, or multiple epitopes from two or more different target antigens (polytope vaccines) originating from the same type of target antigens (e.g., both antigens are peptides or both antigens are glycolipids), or different types of target antigens (e.g., glycolipid antigen and peptide antigen).

Vaccine compositions of the present invention may be administered by any known route of administration. Preferably the compositions of the present invention are administered via the respiratory tract, e.g., by intratracheal instillation or intranasal inhalation. In one embodiment, the compositions of the present invention are administered via intramuscular or subcutaneous injection and in this manner larger-sized (150-300 nm) lipid particles can be used. Consequently, the need for costly extrusion steps can be reduced or eliminated, and since the particles do not need to circulate, the selection of lipid components can be biased in favor of less expensive materials. For example, the amount of Chol can be reduced, DSPC can be replaced with something less rigid (e.g., DOPC or DMPC), and PEG-lipids can be replaced with less expensive PEG-acyl chains.

Immunotherapy or vaccination protocols for priming, boosting, and maintenance of dosing are well known in the art and further described below.

Manufacturing of Compositions

Manufacturing the compositions of the invention may be accomplished by any technique, but most preferred are the ethanol dialysis or detergent dialysis methods detailed in the following publications, patents, and applications each incorporated herein by reference: U.S. Pat. No. 5,705,385; U.S. Pat. No. 5,976,567; U.S. patent application Ser. No. 09/140,476; U.S. Pat. No. 5,981,501; U.S. Pat. No. 6,287,591; Int. Publ. No. WO 96/40964; and Int. Publ. No. WO 98/51278. These manufacturing methods provide for small and large scale manufacturing of immunostimulatory compositions comprising therapeutic agents encapsulated in a lipid particle, preferably lipid-nucleic acid particles. The methods also generate such particles with excellent pharmaceutical characteristics.

Vaccine compositions of the present invention may be prepared by adding a target antigen (to which the immune response is desired). Means of incorporating antigens are well known in the art and include, for example: 1) passive encapsulation of the antigen during the formulation process (e.g., the antigen can be added to the solution containing the ODN); 2) addition of glycolipids and other antigenic lipids to an ethanol lipid mixture and formulated using the ethanol-based protocols described herein; 3) insertion into the lipid vesicle (e.g., antigen-lipid can be added into formed lipid vesicles by incubating the vesicles with antigen-lipid micelles); and 4) the antigen can be added post-formulation (e.g., coupling in which a lipid with a linker moiety is included into formulated particle, and the linker is activated post formulation to couple a desired antigen). Standard coupling and cross-linking methodologies are well known in the art. An alternative preparation incorporates the antigen into a lipid-particle which does not contain a nucleic acid, and these particles are mixed with lipid-nucleic acid particles prior to administration to the subject.

Characterization of Compositions Used in the Methods of the Present Invention.

Preferred characteristics of the compositions used in the the methods of the present invention are as follow.

The lipid-nucleic acid particles of the invention comprise a lipid membrane (generally a phospholipid bilayer) exterior which fully encapsulates an interior space. These particles, also sometimes herein called lipid membrane vesicles, are small particles with mean diameter 50-200 nm, preferably 60-130 nm. Most preferred for intravenous administrations are particles of a relatively uniform size wherein 95% of particles are within 30 nm of the mean. The nucleic acid and other bioactive agents are contained in the interior space, or associated with an interior surface of the encapsulating membrane.

“Fully encapsulated” as used herein indicates that the nucleic acid in the particles is not significantly degraded after exposure to serum or a nuclease assay that would significantly degrade free DNA. In a fully encapsulated system, preferably less than 25% of particle nucleic acid is degraded in a treatment that would normally degrade 100% of free nucleic acid, more preferably less than 10% and most preferably less than 5% of the particle nucleic acid is degraded. Alternatively, full encapsulation may be determined by an Oligreen™ assay. Fully encapsulated also suggests that the particles are serum stable, that is, that they do not rapidly decompose into their component parts upon in vivo administration.

These characteristics of the compositions of the present invention distinguish the key particles of the invention from lipid-nucleic acid aggregates (also known as cationic complexes or lipoplexes) such as DOTMA/DOPE (LIPOFECTIN™) formulations. These aggregates are generally much larger (>250 nm) diameter, they do not competently withstand nuclease digestion, and they generally decompose upon in vivo administration. Formulations of cationic lipid-nucleic acid aggregates with weak antigens, as described above, may provide suitable vaccines for local and regional applications, such as intramuscular, intra-peritoneal and intrathecal administrations, and more preferably intranasal administration.

The particles of the invention can be formulated at a wide range of drug:lipid ratios. “Drug to lipid ratio” as used herein refers to the amount of therapeutic nucleic acid (i.e., the amount of nucleic acid which is encapsulated and which will not be rapidly degraded upon exposure to the blood) in a defined volume of preparation divided by the amount of lipid in the same volume. This may be determined on a mole per mole basis or on a weight per weight basis, or on a weight per mole basis. Drug to lipid ratio may determine the lipid dose that is associated with a given dose of nucleic acid. In a preferred embodiment, the compositions of the present invention have a drug:lipid ratio in the range of about 0.01 to 0.25 (wt/wt).

Uses of the Compositions and Methods of the Present Invention

The present invention provides immunostimulatory compositions and methods of using such compositions to stimulate immune responses in mammals. Particularly, the present invention provides immunostimulatory lipid-nucleic acid (“LNA”) formulations and methods of using such formulations to stimulate immune responses in mammals, and more particularly, mucosal immune responses. The present invention further provides immunostimulatory LNA formulations comprising antigens, and methods of using such formulations to stimulate mucosal immune responses to target antigens or pathogens in mammals. The LNA formulations of the present invention can further comprise additional therapeutic agents useful for treating a disease or disorder in a patient.

In a preferred embodiment, the vaccine compositions of the present invention stimulate an immune response directed to a pathogen. Examples of such pathogens are, but not limited to, HIV, HPV, HSV-1, HSV-2, Neisseria gonorrhea, Chlamydia, and Treponema pallidum can provide antigens or DNA sequences encoding antigens for use in the methods of this invention. Thus, additional antigens suitable for use in the present invention include, but are not limited to, the L1 protein of HPV, the L2 protein of HPV, the E6 protein of HPV, the E7 protein of HPV, the gp41 protein of HIV, the gag protein of HIV, the tet protein of HIV and the gp120 glycoprotein of HIV, among others. Still other pathogens for which such vaccines and vaccine protocols of the present invention are useful include, but are not limited to, the pathogens that cause trichomoniasis, candidiasis, hepatitis, scabies, and syphilis. Further, pathogens which invade via the mucosa also include, but are limited to, those that cause respiratory syncytial virus, flu, other upper respiratory conditions, as well as agents which cause intestinal infections. The methods of stimulating mucosal immunity provided herein are readily applicable to vaccine protocols of vaccines to any pathogen against which mucosal immunity is effective. Further, the invention encompasses the expression of antigens derived from a wide range of human pathogens to which mucosal immunity is desired. Thus, the invention is not limited by the identity of a particular antigen.

As mentioned, the stimulation of an immune response can be broadly characterized as a direct or indirect response of a cell or component of the immune system to an intervention. These responses can be measured in many ways including activation, proliferation or differentiation of cells of the immune system (e.g., B cells, T cells, dendritic cells, APCs, macrophages, NK cells, NKT cells etc.); up-regulated or down-regulated expression of markers; cytokine; interferon; stimulation in IgA, IgM, or IgG titer; splenomegaly (including increased spleen cellularity); znc hyperplasia and mixed cellular infiltrates in various organs. Other responses, cells, and components of the immune system which can be assessed with respect to immune stimulation are known in the art. Further, the stimulation or response may be of innate cells of the immune system, or of acquired cells of the immune system (e.g., as by a vaccine containing a normally weak antigen).

In a preferred embodiment, the compositions and methods of the present invention can be used to modulate the level of a cytokine. “Modulate” as used herein with reference to a cytokine may refer to the suppression of expression of a particular cytokine when lower levels are desired, or augmentation of the expression of a particular cytokine when higher levels are desired. Modulation of a particular cytokine can occur locally or systemically. In a preferrred embodiment, the compositions and methods of the present invention can be used to activate macrophages and dendritic cells to secrete cytokines. It is known that cytokine profiles can determine T cell regulatory and effector functions in immune responses. In general, Th1-type cytokines can be induced, thus the immunostimulatory compositions of the present invention can promote a Th1 type antigen-specific immune response including cytotoxic T-cells.

Cytokines also play a role in directing the T cell response. Helper (CD4⁺) T cells orchestrate the immune response of mammals through production of soluble factors that act on other immune system cells, including B and other T cells. Most mature CD4.sup.+T helper cells express one of two cytokine profiles: Th1 or Th2. Th1 cells secrete IL-2, IL-3, IFN-γ, GM-CSF and high levels of TNF-α Th2 cells express IL-3, IL-4, IL-5, IL-6, IL-9, IL-10, IL-13, GM-CSF and low levels of TNF-α. The Th1 subset promotes both cell-mediated immunity, and humoral immunity that is characterized by immunoglobulin class switching to IgG2a in mice. Th1 responses may also be associated with delayed-type hypersensitivity and autoimmune disease. The Th2 subset induces primarily humoral immunity and induce class switching to IgG₁, and IgE. The antibody isotypes associated with Th1 responses generally have good neutralizing and opsonizing capabilities whereas those associated with Th2 responses are associated more with allergic responses.

Several factors have been shown to influence commitment to Th1 or Th2 profiles. The best characterized regulators are cytokines. IL-12 and IFN-.gamma. are positive Th1 and negative Th2 regulators. IL-12 promotes IFN-γ production, and IFN-γ provides positive feedback for IL-12. IL-4 and IL-10 appear to be required for the establishment of the Th2 cytokine profile and to down-regulate Th1 cytokine production; the effects of IL-4 are in some cases dominant over those of IL-12. IL-13 was shown to inhibit expression of inflammatory cytokines, including IL-12 and TNF-.alpha. by LPS-induced monocytes, in a way similar to IL-4. The IL-12 p40 homodimer binds to the IL-12 receptor and may antagonizes IL-12 biological activity; thus it blocks the pro-Th1 effects of IL-12 in some animals.

In a preferred embodiment, the methods of the present invention comprise stimulating a T Helper 1 cell (“Th1”) immune response in a subject by administering to the subject an effective amount of the immunostimulatory compositions of the present invention. Preferably the immunostimulatory compositions are LNA formulations comprising an ODN. In a preferred embodiment, the methods of the present invention comprise stimulating a T Helper 2 cell (“Th2”) immune response in a subject by administering to the subject an effective amount of the immunostimulatory compositions of the present invention. Preferably the immunostimulatory compositions are LNA formulations comprising an ODN. As described above a Th2 profile is characterized by production of IL-4 and IL-10. Non-nucleic acid adjuvants that induce Th2 or weak Th1 responses include but are not limited to alum, saponins, oil-in-water and other emulsion formulations and SB-As4. Adjuvants that induce Th1 responses include but are not limited to MPL, MDP, ISCOMS, IL-12, IFN-γ, and SB-AS2.

Antigens may be used in the compositions and methods of the present invention in a crude, purified, synthetic, isolated, or recombinant form. Polypeptide or peptide antigens, (including, for example, antigens that are peptide mimics of polysaccharides) encoded by nucleic acids may also be used in the compositions and methods of the present invention. The term antigen broadly includes any type of molecule which is recognized by a host immune system as being foreign. Antigens include but are not limited to cancer antigens, microbial antigens, and allergens.

A “cancer antigen” as used herein is a compound (e.g., a peptide) associated with a tumor or cancer cell surface and which is capable of provoking an immune response when expressed on the surface of an antigen presenting cell in the context of an MHC molecule. Cancer antigens can be prepared by methods known in the art. For example, cancer antigens can be prepared from cancer cells either by preparing crude extracts of cancer cells (e.g., as described in Cohen, et al., 1994, Cancer Research, 54:1055), by partially purifying the antigens, by recombinant technology, or by de novo synthesis of known antigens. Examples of cancer antigens include, but are not limited to, antigens that are an immunogenic portion of or a whole tumor or cancer. Such antigens can be isolated or prepared recombinantly or by any other means known in the art.

A “microbial antigen” as used herein is an antigen of a microorganism and includes but is not limited to, infectious virus, infectious bacteria, infectious parasites and infectious fungi. Examples of microbial antigens include, but are not limited to, intact microorganisms, and natural isolates, fragments, or derivatives thereof, synthetic compounds which are identical to or similar to naturally-occuring microbial antigens and, preferably, induce an immune response specific for the corresponding microorganism (from which the naturally-occuring microbial antigen originated). In a preferred embodiment, a compound is similar to a naturally-occuring microorganism antigen if it induces an immune response (humoral and/or cellular) to a naturally-occuring microorganism antigen. Compounds or antigens that are similar to a naturally-occuring microorganism antigen are well known to those of ordinary skill in the art. A nonlimiting example of a compound that is similar to a naturally-occuring microorganism antigen is a peptide mimic of a polysaccharide antigen.

Examples of pathogens include, but are not limited to, infectious virus that infect mammals, and more particularly humans. Examples of infectious virus include, but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Also, gram negative and gram positive bacteria serve as antigens in vertebrate animals. Such gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus infuenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of pathogens include, but are not limited to, infectious fungi that infect mammals, and more particularly humans. Examples of infectious fungi include, but are not limited to: Cryptococcus neoformans, Histoplasma capsulatum, Coccidioides immitis, Blastomyces dermatitidis, Chlamydia trachomatis, Candida albicans. Examples of infectious parasites include Plasmodium such as Plasmodium falciparum, Plasmodium malariae, Plasmodium ovale, and Plasmodium vivax. Other infectious organisms (i.e. protists) include Toxoplasma gondii.

Other medically relevant microorganisms that serve as antigens in mammals and more particularly humans are described extensively in the literature, e.g., see C. G. A Thomas, Medical Microbiology, Bailliere Tindall, Great Britain 1983, the entire contents of which is hereby incorporated by reference. In addition to the treatment of infectious human diseases, the compositions and methods of the present invention are useful for treating infections of nonhuman mammals.

In preferred embodiments, “treatment”, “treat”, “treating” as used herein with reference to infectious pathogens, refers to a prophylactic treatment which increases the resistance of a subject to infection with a pathogen or decreases the likelihood that the subject will become infected with the pathogen; and/or treatment after the subject has become infected in order to fight the infection, e.g., reduce or eliminate the infection or prevent it from becoming worse. Many vaccines for the treatment of non-human mammals are disclosed in Bennett, K. Compendium of Veterinary Products, 3rd ed. North American Compendiums, Inc., 1995. As discussed above, antigens include infectious microbes such as virus, bacteria, parasites and fungi and fragments thereof, derived from natural sources or synthetically. Infectious virus of both human and non-human mammals, include retroviruses, RNA viruses, and DNA viruses. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (“MMTV”). The C-type retroviruses include subgroups C-type group A (including Rous sarcoma virus (“RSV”), avian leukemia virus (“ALV”), and avian myeloblastosis virus (“AMV”)) and C-type group B (including murine leukemia virus (“MLV”), feline leukemia virus (“FeLV”), murine sarcoma virus (“MSV”), gibbon ape leukemia virus (“GALV”), spleen necrosis virus (“SNV”), reticuloendotheliosis virus (“RV”) and simian sarcoma virus (“SSV”). The D-type retroviruses include Mason-Pfizer monkey virus (“MPMV”) and simian retrovirus type 1 (“SRV-1”). The complex retroviruses include the subgroups of lentiviruses, T-cell leukemia viruses and the foamy viruses. Lentiviruses include HIV-1, but also include HIV-2, SIV, Visna virus, feline immunodeficiency virus (“FIV”), and equine infectious anemia virus (“EIAV”). The T-cell leukemia viruses include HTLV-1, HTLV-II, simian T-cell leukemia virus (“STLV”), and bovine leukemia virus (“BLV”). The foamy viruses include human foamy virus (“HFV”), simian foamy virus (“SFV”) and bovine foamy virus (“BFV”).

Examples of other RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (“ECHO”) viruses, hepatitis A virus, Simian enteroviruses, Murine encephalomyelitis (“ME”) viruses, Poliovirus muris, Bovine enteroviruses, Porcine enteroviruses, the genus Cardiovirus (Encephalomyocarditis virus (“EMC”), Mengovirus), the genus Rhinovirus (Human rhinoviruses including at least 113 subtypes; other rhinoviruses), the genus Apthovirus (Foot and Mouth disease (“FMDV”); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (“RSV”), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (“RSV”), Bovine respiratory syncytial virus and Pneumonia virus of mice); the family Rhabdoviridae, including the genus Vesiculovirus (“VSV”), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (“LCM”), Tacaribe virus complex, and Lassa virus; the family Coronoaviridae, including Infectious Bronchitis Virus (“IBV”), Mouse Hepatitis virus, Human enteric corona virus, and Feline infectious peritonitis (Feline coronavirus).

Illustrative DNA viruses that are antigens in vertebrate animals include, but are not limited to: the family Poxyiridae, including the genus Orthopoxyirus (Variola major, Variolaminor, Monkey pox Vaccinia, Cowpox, Buffalopox, Rabbitpox, Ectromelia), the genus Leporipoxyirus (Myxoma, Fibroma), the genus Avipoxyirus (Fowlpox, other avian poxyirus), the genus Capripoxyirus (sheeppox, goatpox), the genus Suipoxyirus (Swinepox), the genus Parapoxyirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesvirises (Human cytomegalovirus and cytomegaloviruses of swine, monkeys and rodents); the gamma-herpesviruses (Epstein-Barr virus (“EBV”), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (“SV-40”), Rabbit vacuolating agent (“RKV”), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus). Finally, DNA viruses may include viruses which do not fit into the above families such as Kuru and Creutzfeldt-Jacob disease viruses and chronic infectious neuropathic agents (CHINA virus).

In addition to the use of nucleic acid and non-nucleic acid adjuvants to stimulate an antigen-specific immune response in mammals, the methods of the preferred embodiments are particularly well suited for treatment of other vertebrates, for example, birds such as hens, chickens, turkeys, ducks, geese, quail, and pheasant. Birds are prime targets for many types of infectious pathogens.

An example of a common infection in chickens is chicken infectious anemia virus (“CIAV”). CIAV was first isolated in Japan in 1979 during an investigation of a Marek's disease vaccination break (Yuasa et al., 1979, Avian Dis. 23:366-385). Since that time, CIAV has been detected in commercial poultry in all major poultry producing countries (van Bulow et a., 1991, pp.690-699) in Diseases of Poultry, 9th edition, Iowa State University Press).

CIAV infection results in a clinical disease, characterized by anemia, hemorrhage and immunosuppression, in young susceptible chickens. Atrophy of the thymus and of the bone marrow and consistent lesions of CIAV-infected chickens are also characteristic of CIAV infection. Lymphocyte depletion in the thymus, and occasionally in the bursa of Fabricius, results in immunosuppression and increased susceptibility to secondary viral, bacterial, or fungal infections which then complicate the course of the disease. The immunosuppression may cause aggravated disease after infection with one or more of Marek's disease virus (“MDV”), infectious bursal disease virus, reticuloendotheliosis virus, adenovirus, or reovirus. It has been reported that pathogenesis of MDV is enhanced by CIAV (DeBoer et al., 1989, p. 28 In Proceedings of the 38th Western Poultry Diseases Conference, Tempe, Ariz.). Further, it has been reported that CIAV aggravates the signs of infectious bursal disease (Rosenberger et a., 1989, Avian Dis. 33:707-713). Chickens develop an age resistance to experimentally induced disease due to CIAV which is essentially complete by the age of 2 weeks, but older birds are still susceptible to infection (Yuasa, N. et a., 1979 supra; Yuasa, N. et al., Arian Diseases 24, 202-209, 1980). However, if chickens are dually infected with CIAV and an immunosuppressive agent (IBDV, MDV etc.) age resistance against the disease is delayed (Yuasa, N. et a., 1979 and 1980 supra; Bulow von V. et al., J. Veterinary Medicine 33, 93-116, 1986). Characteristics of CIAV that may potentiate disease transmission include, for example, high resistance to environmental inactivation and some common disinfectants.

Vaccination of birds, like other vertebrate animals can be performed at any age. Normally, vaccinations are performed at up to 12 weeks of age for a live microorganism and between 14-18 weeks for an inactivated microorganism or other type of vaccine. For in ovo vaccination, vaccination can be performed in the last quarter of embryo development. The vaccine may be administered subcutaneously, by spray, orally, intraocularly, intratracheally, nasally, in ovo or by other methods described herein. Thus, the compositions of the present invention can be administered to birds and other non-human vertebrates using routine vaccination schedules and the antigen is administered after an appropriate time period as described herein.

Cattle and livestock are also susceptible to infection. Disease which affect these animals can produce severe economic losses, especially amongst cattle. The methods of the present invention can be used to protect against infection in livestock, such as cows, horses, pigs, sheep, and goats. The compositions of the present invention could also be administered with antigen for antigen-specific protection of long duration or without antigen for short term protection against a wide variety of diseases, including shipping fever.

Cows can be infected by, for example, bovine viruses. Bovine viral diarrhea virus (“BVDV”) is a small enveloped positive-stranded RNA virus and is classified, along with hog cholera virus (“HOCV”) and sheep border disease virus (BDV), in the pestivirus genus. Although, Pestiviruses were previously classified in the Togaviridae family, some studies have suggested their reclassification within the Flaviviridae family along with the flavivirus and hepatitis C virus (“HCV”) groups (Francki, et al., 1991).

BVDV, which is an important pathogen of cattle can be distinguished, based on cell culture analysis, into cytopathogenic (“CP”) and noncytopathogenic (“NCP”) biotypes. The NCP biotype is more widespread although both biotypes can be found in cattle. If a pregnant cow becomes infected with an NCP strain, the cow can give birth to a persistently infected and specifically immunotolerant calf that will spread virus during its lifetime. The persistently infected cattle can succumb to mucosal disease and both biotypes can then be isolated from the animal. Clinical Manifestations can include abortion, teratogenesis, and respiratory problems, mucosal disease and mild diarrhea. In addition, severe thrombocytopenia, associated with herd epidemics, that may result in the death of the animal has been described and strains associated with this disease seem more virulent than the classical BVDVs.

Equine herpesviruses (“EHV) comprise a group of antigenically distinct biological agents which cause a variety of infections in horses ranging from subclinical to fatal disease. These include Equine herpesvirus-1 (“EHV-1”), a ubiquitous pathogen in horses. EHV-1 is associated with epidemics of abortion, respiratory tract disease, and central nervous system disorders. Primary infection of upper respiratory tract of young horses results in a febrile illness which lasts for 8 to 10 days. Immunologically experienced mares may be reinfected via the respiratory tract without disease becoming apparent, so that abortion usually occurs without warning. The neurological syndrome is associated with respiratory disease or abortion and can affect animals of either sex at any age, leading to incoordination, weakness and posterior paralysis (Telford, E. A. R. et a., Virology 189, 304-316, 1992). Other EHV's include EHV-2, or equine cytomegalovirus, EHV-3, equine coital exanthema virus, and EHV-4, previously classified as EHV-1 subtype 2.

Sheep and goats can be infected by a variety of dangerous microorganisms including visna-maedi.

Primates such as monkeys, apes and macaques can be infected by simian immunodeficiency virus (“SIV”). Primates infected by SIV are known to be responsive to some vaccines (Stott et al. (1990) Lancet 36:1538-1541; Desrosiers et al. PNAS USA (1989) 86:6353-6357; Murphey-Corb et al (1989) Science 246:1293-1297; and Carlson et a. (1990) AIDS Res. Human Retroviruses 6:1239-1246; Berman et al. (1990) Nature 345:622-625).

Cats, both domestic and wild, are susceptible to infection with a variety of microorganisms. For example, feline infectious peritonitis is a disease which occurs in both domestic and wild cats, such as lions, leopards, cheetahs, and jaguars. When it is desirable to prevent infection with this and other types of pathogenic organisms in cats, the methods of the invention can be used to vaccinate cats to prevent them against infection.

Domestic cats may become infected with several retroviruses, including but not limited to feline leukemia virus (FeLV), feline sarcoma virus (FeSV), endogenous type C oncornavirus (RD-114), and feline syncytia-forming virus (FeSFV). Of these, FeLV is the most significant pathogen, causing diverse symptoms, including lymphoreticular and myeloid neoplasms, anemias, immune mediated disorders, and an immunodeficiency syndrome which is similar to human acquired immune deficiency syndrome (AIDS). Recently, a particular replication-defective FeLV mutant, designated FeLV-AIDS, has been more particularly associated with immunosuppressive properties.

The discovery of feline T-lymphotropic lentivirus (also referred to as feline immunodeficiency) was first reported by Pedersen et al. (1987) Science 235:790-793. Characteristics of FIV have been reported in Yamamoto et al. (1988) Leukemia, December Supplement 2:204S-215S; Yamamoto et al. (1988) Am. J. Vet. Res. 49:1246-1258; and Ackley et al. (1990) J. Virol. 64:5652-5655. Cloning and sequence analysis of FIV have been reported in Olmsted et al. (1989) Proc. Natl. Acad. Sci. USA 86:2448-2452 and 86:4355-4360.

Feline infectious peritonitis (FIP) is a sporadic disease occurring unpredictably in domestic and wild Felidae. While FIP is primarily a disease of domestic cats, it has been diagnosed in lions, mountain lions, leopards, cheetahs, and the jaguar. Smaller wild cats that have been afflicted with FIP include the lynx and caracal, sand cat, and pallas cat. In domestic cats, the disease occurs predominantly in young animals, although cats of all ages are susceptible. A peak incidence occurs between 6 and 12 months of age. A decline in incidence is noted from 5 to 13 years of age, followed by an increased incidence in cats 14 to 15 years old.

Viral, bacterial and parasitic diseases in fin-fish, shellfish or other aquatic life forms pose a serious problem for the aquaculture industry. Owing to the high density of animals in the hatchery tanks or enclosed marine farming areas, infectious diseases may eradicate a large proportion of the stock in, for example, a fin-fish, shellfish, or other aquatic life forms facility. Prevention of disease is a more desired remedy to these threats to fish than intervention once the disease is in progress. Vaccination of fish is the only preventative method which may offer long-term protection through immunity. Fish are currently protected against a variety of bacterial infections with whole killed vaccines with oli adjuvants, but there is only one licensed vaccine for fish against a viral disease. Nucleic acid based vaccinations are described in U.S. Pat. No. 5,780,448 issued to Davis and these have been shown to be protective against at least two different viral diseases.

The fish immune system has many features similar to the mammalian immune system, such as the presence of B cells, T cells, lymphokines, complement, and immunoglobulins. Fish have lymphocyte subclasses with roles that appear similar in many respects to those of the B and T cells of mammals. Vaccines can be administered orally or by immersion or injection.

Aquaculture species include but are not limited to fin-fish other aquatic animals. Fin-fish include all vertebrate fish, which may be bony or cartilaginous fish, such as, for example, salmonids, carp, catfish, yellowtail, seabream, and seabass. Salmonids are a family of fin-fish which include trout (including rainbow trout), salmon, and Arctic char. Polypeptides of viral aquaculture pathogens include but are not limited to glycoprotein (“G protein”) or nucleoprotein (“N protein”) of viral hemorrhagic septicemia virus (“VHSV”); G or N proteins of infectious hematopoietic necrosis virus (“IHNV”); VP1, VP2, VP3 or N structural proteins of infectious pancreatic necrosis virus (“IPNV”); G protein of spring viremia of carp (“SVC”); and a membrane-associated protein, tegumin or capsid protein or glycoprotein of channel catfish virus (“CCV”).

Polypeptides of bacterial pathogens include but are not limited to an iron-regulated outer membrane protein, (“IROMP”), an outer membrane protein (“OMP”), and an A-protein of Aeromonis salmonicida which causes furunculosis, p57 protein of Renibacterium salmoninarum which causes bacterial kidney disease (“BKD”), major surface associated antigen (“msa”), a surface expressed cytotoxin (“mpr”), a surface expressed hemolysin (“ish”), and a flagellar antigen of Yersiniosis; an extracellular protein (“ECP”), an iron-regulated outer membrane protein (“IROMP”), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda; and surface antigen of Ichthyophthirius; and a structural and regulatory protein of Cytophaga columnari; and a structural and regulatory protein of Rickettsia.

Polypeptides of a parasitic pathogen include but are not limited to the surface antigens of lchthyophthirius.

An “allergen” refers to a substance (antigen) that can induce an allergic or asthmatic response in a susceptible subject. The list of allergens is enormous and can include pollens, insect venoms, animal dander dust, fungal spores and drugs (e.g. penicillin). Examples of natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. PArrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

The compositions and methods of the present invention can be used for immunizing an infant by administering to an infant an immunostimulatory composition of the present invention in an effective amount for inducing cell mediated immunity in the infant. In some embodiments the infant is also administered at least one non-nucleic acid adjuvant, as described above. Cell mediated immunity, as used herein, refers to an immune response which involves an antigen specific T cell reaction. The presence of cell mediated immunity can be determined directly by the induction of Th1 cytokines (e.g., IFN-.gamma., IL-12) and antigen-specific cytotoxic T-cell lymphocytes (CTL). The presence of cell mediated immunity is also indicated indirectly by the isotype of antigen-specific antibodies that are induced (e.g., IgG2a, IgG1 in mice). Thus, if Th1 cytokines or CTL or TH2-like antibodies are induced, cell mediated immunity is induced according to the invention. As discussed above, Th1 cytokines include but are not limited to IL-12 and IFN-.gamma.

Neonates (newborn) and infants (which include humans three months of age and referred to hereinafter as infants) born in HBV endemic areas require particularly rapid induction of strong HBV-specific immunity owing to the high rate of chronicity resulting from infection at a young age. Without immunoprophylaxis, 70-90% of infants born to mothers positive for both HBsAg and the “e” antigen (HBeAg) become infected and almost all of these become chronic carriers (Stevens et al., 1987). Even when vaccinated with a four dose regime of the HBV subunit vaccine commencing on the day of birth, 20% of such infants became chronically infected and this was reduced to only 15% if they were also given HBV-specific immunoglobulin (Chen et al. 1996) HBV chronicity results in 10-15% of individuals infected as adolescents or adults, but 90-95% for those infected (either vertically or horizontally) as infants. The compositions of the present invention could be prepared with HBe antigen and used in the methods of the present invention further reduce such chronic infections owing to a more rapid appearance and higher titers of anti-HB antibodies and the induction of HBV-specific CTL, which could help clear virus from the liver of babies infected in utero, and which likely account for most of the failures with infant vaccination.

Indications, Administration and Dosages

The compositions and methods of the present invention are indicated for use in any patient or organism having a need for immune system stimulation. Such a need encompasses, but is not limited to, most medical fields, such as oncology, inflammation, arthritis & rheumatology, immuno-deficiency disorders. One skilled in the art can select appropriate indications to test for efficacy based on the disclosure herein. In a preferred embodiment, the compositions and methods of the invention are used to treat a neoplasia (any neoplastic cell growth which is pathological or potentially pathological) such as the neoplasia described in the Examples below.

Administration of the compositions of the invention to a subject may be by any method including in vivo or ex vivo methods. In vivo methods can include local, regional or systemic applications. In a preferred embodiment, the compositions are administered intravenously such that particles are accessible to B cells, macrophages or a splenocytes in a patient, and/or the particle can stimulate lymphocyte proliferation, resulting in secretion of IL-6, IL-12, IFNg and/or IgM in said patient.

One skilled in the art would know how to identify possible toxicities of formulations, for example, complement activation, coagulation, renal toxicities, liver enzyme assays, etc. Such toxicities may differ between organisms.

Pharmaceutical preparations of compositions usually employ additional carriers to improve or assist the delivery modality. Typically, compositions of the invention will be administered in a physiologically-acceptable carrier such as normal saline or phosphate buffer selected in accordance with standard pharmaceutical practice. Other suitable carriers include water, 0.9% saline, 0.3% glycine, and the like, including glycoproteins for enhanced stability, such as albumin, lipoprotein, globulin, etc.

Dosages of lipid-nucleic acid formulations depend on the desired lipid dosage, the desired nucleic acid dosage, and the drug:lipid ratio of the composition. One skilled in the art can select proper dosages based on the information provided herein.

“Effective amount” as used herein refers to the amount necessary or sufficient to realize a desired biologic effect. In preferred embodiments, the biological effect is the stimulation of an immune response, and preferably an immune response. As a nonlimiting example, an effective amount of an LNA formulation or LNA formulation-Ag comprising an ISS ODN for treating an infectious disorder, is that amount necessary to cause the development of an antigen specific immune response upon exposure to the microbe, thereby, causing a reduction in the amount of microbe within the subject, and preferably eradication of the microbe. The effective amount for a particular application can vary depending on such factors as, for example, the disease, disorder, or condition being treated, the particular ISS ODN or other therapeutic agent being administered, the body weight of the subject, or the severity of the disease, disorder, or condition. One of ordinary skill in the art would know how to empirically determine the effective amount of a particular adjuvant and antigen without necessitating undue experimentation.

Immunotherapy or vaccination protocols for priming, boosting, and maintenance of dosing are well known in the art and further described below. In particular, one skilled in the art would know how to calculate dosage amounts for a subject, particularly a mammal, and more particularly a human, based on the dosage amounts described herein. Specific conversion factors for converting dosage amounts from one animal to another (e.g., from mouse to human) are well known in the art and are fully described, e.g., on the Food and Drug Administration Web site at: www.fda.gov/cder/cancer/animalframe.htm (in the oncology tools section), incorporated herein by reference. As compared to known immunostimulatory compositions having free nucleic acids, the immunostimulatory compositions and methods of the present invention may utilize reduced amounts of nucleic acids to stimulate enhanced mucosal immune responses in vivo.

In some embodiments, the amount of nucleic acids in the LNA formulations of the present invention is about about 0.001-60 mg/kg (mg nucleic acids per mg body weight of a mouse). In preferred embodiments, the compositions and methods of the present invention utilize less than about 10 mg/kg (mg nucleic acids per mg body weight of a mouse), more preferably less than about 1 mg/kg (mg nucleic acids per mg body weight of a mouse), most preferably less than about 0.1 mg/kg (mg nucleic acids per mg body weight of a mouse), and optimally less than about 0.01 mg/kg (mg nucleic acids per mg body weight of a mouse). In preferred embodiments, the amount of nucleic acids in the LNA formulations of the present invention is about 0.001-10 mg/kg (mg nucleic acids per mg body weight of a mouse), more preferably about 0.001-1 mg/kg (mg nucleic acids per mg body weight of a mouse), even more preferably about 0.001-0.1 mg/kg (mg nucleic acids per mg body weight of a mouse), and most preferably about 0.001-0.01 mg/kg (mg nucleic acids per mg body weight of a mouse). In some embodiments, the amount of antigen associated with the LNA formulations of the present invention is about about 0.004-40 mg/kg (mg antigen per mg body weight of a mouse). In preferred embodiments, the compositions and methods of the present invention the amount of antigen associated with the LNA formulations of the present invention is about 0.004-4 mg/kg (mg antigen per mg body weight of a mouse). As described above, one skilled in the art could readily determine suitable dosage amounts for other mammals given the dosage amounts described herein, based on the well-known conversion factors identified above and further empirical testing.

The formulations of the invention may be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the immunostimulatory compositions of the present invention can be administered to a subject by any mode allowing uptake by the appropriate target cells. “Administering” the immunostimulatory composition of the present invention may be accomplished by any means known to the skilled artisan. Preferred routes of administration include but are not limited to mucosal intranasal, intratracheal, inhalation, and intrarectal, intravaginal; or oral, transdermal (e.g., via a patch), parenteral injection (subcutaneous, intradermal, intravenous, parenteral, intraperitoneal, intrathecal, etc.). An injection may be in a bolus or a continuous infusion.

For example, the immunostimulatory compositions of the present invention can be administered by intramuscular or intradermal injection, or other parenteral means, or by biolistic “gene-gun” application to the epidermis. The immunostimulatory compositions of the present invention may also be administered, for example, by inhalation, topically, intravenously, orally, implantation, rectally, or vaginally. Suitable liquid or solid pharmaceutical preparation forms are, for example, aqueous or saline solutions for injection or inhalation, encochleated, coated onto microscopic gold particles, and nebulized. For a brief review of present methods for drug delivery, see Langer, Science 249:1527-1533, 1990, which is incorporated herein by reference.

The pharmaceutical compositions are preferably prepared and administered in dose units. Liquid dose units are vials or ampoules for injection or oth er parenteral administration. Solid dose units are tablets, capsules and suppositories. For treatment of a patient, depending on activity of the compound, manner of administration, purpose of the immunization (i.e., prophylactic or therapeutic), nature and severity of the disorder, age and body weight of the patient, different doses may be necessary. The administration of a given dose can be carried out both by single administration in the form of an individual dose unit or else several smaller dose units. Multiple administration of doses at specific intervals of weeks or months apart is usual for boosting the antigen-specific responses.

The immunostimulatory compositions of the present invention may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

In preferred embodiments, the immunostimulatory compositions of the present invention contain an effective amount of a combination of adjuvants and antigens optionally included in a pharmaceutically-acceptable carrier. “Pharmaceutically-acceptable carrier” as used herein refers to one or more compatible solid or liquid filler, dilutants or encapsulating substances which are suitable for administration to a human or other mammal. “Carrier” as used herein refers to an organic or inorganic ingredient, natural or synthetic, with which the active ingredient is combined to facilitate the application. The components of the immunostimulatory compositions of the present invention also are capable of being comingled with the compounds of the present invention, and with each other, in a manner such that there is no interaction which would substantially impair the desired pharmaceutical efficiency.

Compositions suitable for parenteral administration conveniently comprise sterile aqueous preparations, which can be isotonic with the blood of the recipient. Among the acceptable vehicles and solvents are water, Ringer's solution, phosphate buffered saline and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for subcutaneous, intramuscular, intraperitoneal, intravenous, etc. administrations may be found in Remington's Pharmaceutical Sciences, Mack Publishing Company, Easton, Pa.

The adjuvants or antigens useful in the invention may be delivered in mixtures of more than two adjuvants or antigens. A mixture may consist of several adjuvants in addition to the synergistic combination of adjuvants or several antigens.

A variety of administration routes are available. The particular mode selected will depend, of course, upon the particular adjuvants or antigen selected, the age and general health status of the subject, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed above.

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within amatrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

The following Examples are illustrative of the disclosed composition and methods, and are not intended to limit the scope of the invention. Without departing from the spirit and scope of the invention, various changes and modifications of the invention will be clear to one skilled in the art and can be made to adapt the invention to various uses and conditions. Thus, other embodiments are encompassed.

### EXAMPLES

### Example 1

Stimulation of an Antigen-Specific Mucosal Immune Response Using LNA formulations

This example illustrates the stimulation of IgA and IgG immune responses to lipid-nucleic acid (LNA) formulations, including LNA formulations comprising a target antigen, chicken ovalbumin (“OVA”).

Oligonucleotides

The oligodeoxyonucleotides (“ODNs”) used in this study were synthesized with a phosphorothioate (“PS”) backbone. The sequences of each ODN are as follows:

| | | |
|---|---|---|
| ODN #1 PS | 5′-TCCATGACGTTCCTGACGTT-3′ | SEQ ID NO:1 |
| ODN #2 PS | 5′-TAACGTTGAGGGGCAT-3′ | SEQ ID NO:2 |
| ODN #3 PS | 5′-TAAGCATACGGGGTGT-3′ | SEQ ID NO:3 |

Preparation of LNA Formulations

LNA formulations were prepared using ODN #1 PS, ODN #2 PS, or ODN #3 PS and OVA as a target antigen. Specifically, LNA formulations were prepared by formulating the ODNs with the lipid mixture DODAP:DSPC:CH:PEG-C14 (25:20:45:10 mol %), using the eth anol-based procedure described in U.S. Pat. No. 6,287,591 incorporated herein by reference. Thereby, liposomes encapsulating ODN #1 PS or ODN #2 PS, or ODN #3 PS were prepared. The particle size of the resulting liposomes was 100-140 nm.

The oligonucletides ODN #1 PS, ODN #2 PS, and ODN #3 PS were each encapsulated in lipid particles using an ethanol dialysis procedure and an ionizable aminolipid previously described (see, for example, Semple et al., Methods Enzymol. (2000) 313:322-341; Semple et al., Biochem. Biophys. Acta. (2001) 1510(1-2):152-166). The ODNs were then hydrated in 300 mM citrate buffer (pH 4.0) and prewarmed to 80° C. for 5 min (minutes) before formulation to ensure the presence of monomer ODNs. The lipid formulations consisted of DSPC/CH/DODAP/PEG-CerC14 at 20/45/25/10 molar ratios. Each ODN was encapsulated separately by slowly adding the lipid mixture dissolved in ethanol to the citrate solution of ODN to give a final ethanol concentration of 40% (vol/vol). The initial ODN to lipid ratio (wt ODN to wt total lipid) was 0.25. The ODN-lipid mixtures were passed 10 times through two stacked 100 nm polycarbonate filters (Osmonics, Livermore, Calif.) using a thermobarrel extruder (Lipex Biomembranes, Vancouver, BC Canada) maintained at approximately 65° C. Non-encapsulated ODN was then removed from the formulation by an initial 1 hr (hour) dialysis against 300 mM citrate buffer, pH 4.0, before an overnight dialysis against HBS (10 mM Hepes, 145 mM NaCl, pH 7.5) followed by DEAE-sepharose CL-6B anion exchange chromatography. The ODN concentration of the formulations was determined by analysis at 260 nm in a spectrophotometer. The mean diameter and size distribution of the LNA particles were determined using a NICOMP Model 370 submicron particle sizer and was typically 110+/−30 nm.

Immunization and Sample Isolation

C57BL/6 mice (6 weeks old) were immunized with 20 μl of the following test formulations by intranasal administration on day 0 (initial immunization), and days 7, and 14 after the initial immunization.

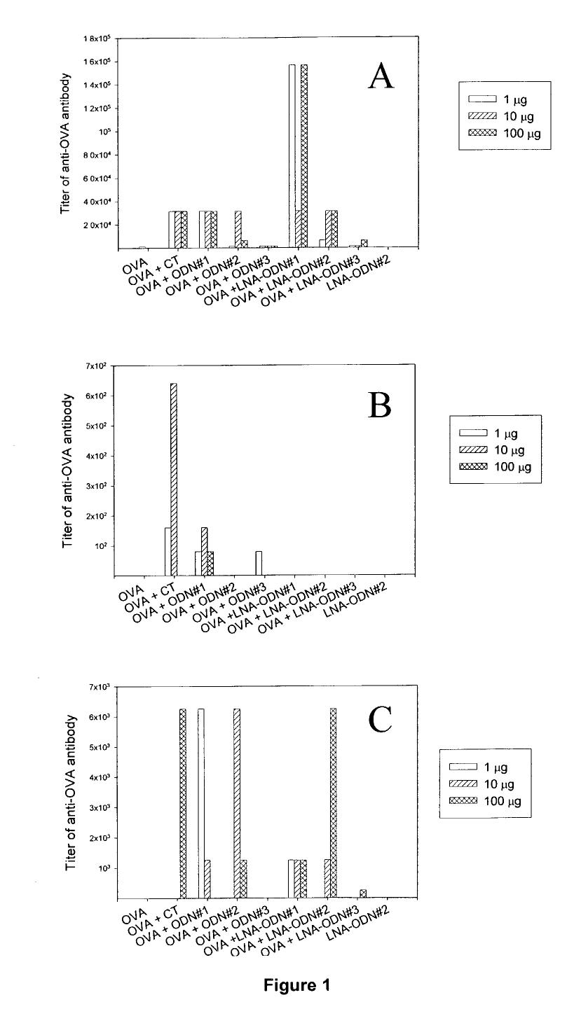

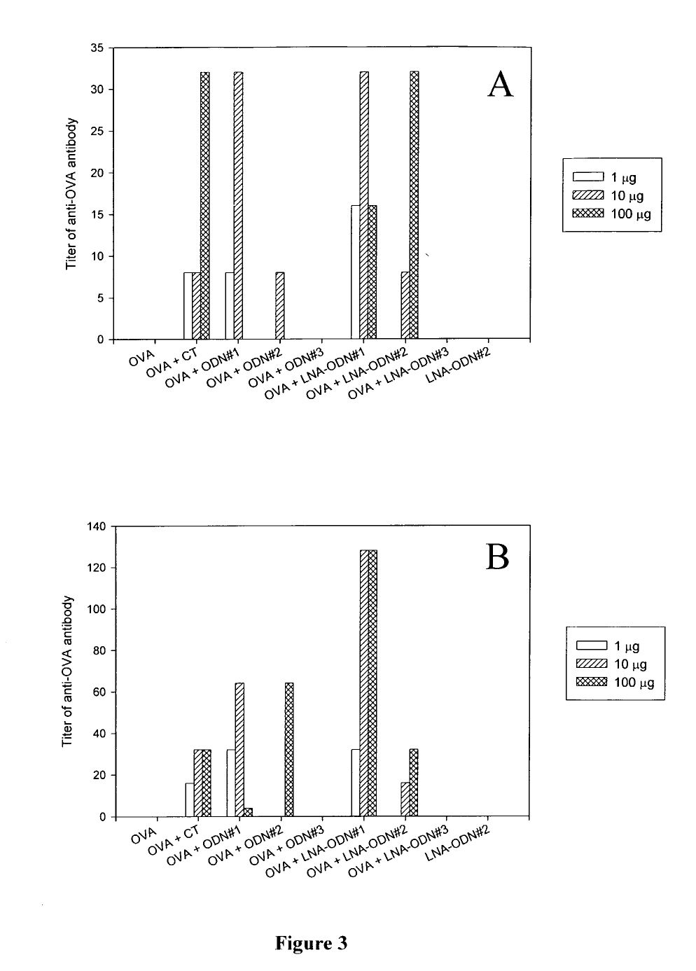

For FIGS. 1-3:

OVA alone

OVA co-administered with 10 μg CT (“OVA+CT”)

OVA co-administered with ODN #1 (“OVA+ODN #1”)

OVA co-administered with ODN #2 (“OVA+ODN #2”)

OVA co-administered with ODN #3 (“OVA+ODN #3”)

OVA co-administered with LNA containing ODN #1 (“OVA+LNA-ODN #1”)

OVA co-administered with LNA containing ODN #2 (“OVA+LNA-ODN #2”)

OVA co-administered* with LNA containing ODN #3 (“OVA+LNA-ODN #3”)

LNA containing ODN #2 (“LNA-ODN #2”)

Mice received OVA protein at a dose of 75 μg per immunization. Free or encapsulated ODN were administered at doses of 1, 10 and 100 μg.

For FIGS. 6 and 7:

PBS alone

OVA co-administered with 10 μg CT (“OVA+CT”)

LNA containing ODN #2 (“LNA-ODN #2”)

OVA co-administered with ODN #1 (“OVA+ODN #1”)

OVA co-administered with LNA containing ODN #1 (“OVA+LNA-ODN #1”)

OVA co-administered with ODN #2 (“OVA+ODN #2”)

OVA co-administered with LNA containing ODN #2 (“OVA+LNA-ODN #2”)

OVA co-administered with ODN #3 (“OVA+ODN #3”)

OVA co-administered* with LNA containing ODN #3 (“OVA+LNA-ODN #3”)

Mice received OVA protein at a dose of 75 μg per immunization. Free or encapsulated ODN were administered at a dose of 100 μg.

Each treatment group (n=5) was anesthetized with halothane before droplets of the vaccine were applied to the esternal nares for complete inhalation. On day 28 after the initial immunization, plasma was collected from anesthetized mice by cardiac puncture and placed into serum tubes. Vaginal washes were obtained by pipetting 50 μl of PBS (Phosphate Buffer Saline) into and out of the vagina of each mouse. This procedure was repeated three times so that a total of 150 μl of washes were collected. The mice were subsequently terminated by cervical dislocation and a lung wash was performed by inserting tubing into the trachea and then pipetting 1 mL of PBS into and out of the lungs. Volume recovery for this precedure was generally 70-80%. Serum tubes were left at room temperature for 30 min to allow for clotting before centrifuging at 10,000 rpm (revolutions per minute) at 4° C. for 5 min, and the resulting aliquots of supernatent collected and stored at −20° C. until analysis. The serum aliquots were also stored at −20° C. until analysis.

ELISA Evaluation of the Immune Response

OVA-specific IgG and IgA antibodies in serum, lung washes, and vaginal washes were measured by ELISA (enzyme-linked immunosorbent assay). Microtiter plates (96 wells) were coated overnight at 4° C. with 5 μl/ml of OVA diluted in PBS (50 μl). The microtiter plates were then washed with PBS containing 0.5% Tween 20 (PBST) and blocked with 200 μl of 1% bovine serum albumin (BSA) in PBST for 1 hr at 37° C. with 50 μl of HRP (horse radish peroxidase)-conjugated goat anti-mouse IgG (1:4000) or HRP-conjugated sheep anti-mouse IgA (1:10) diluted with BSA-PBST. Plates were developed in a 30 min room temperature incubation with TMB (3,3′,5,5′-Tetramethylbenzidine) (100 μL) before stopping the reaction with 50 μl of 0.5 M H₂SO₃. Optical densities were read at 450-570 nm with an ELISA plate reader.

Results

Results are shown in FIGS. 1-3 illustrating antibody titers in serum, lung washes, and vaginal washes.

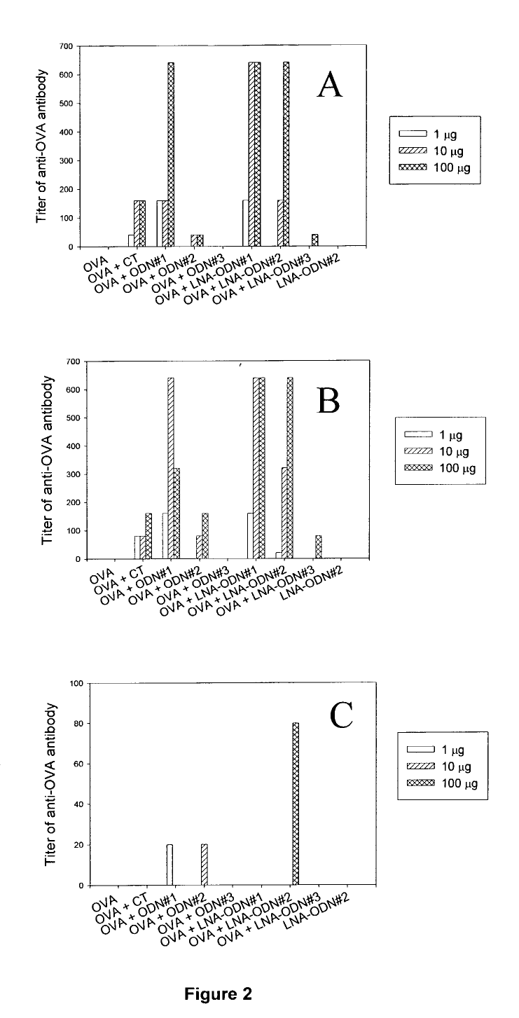

FIGS. 1-3(b) show that, in the test formulations using OVA co-administered with the LNA formulations, anti-OVA-specific IgA levels were increased at both local and distant mucosal sites by several orders of magnitude relative to OVA co-administered with ODN, OVA co-administered with CT, OVA alone, or LNA-ODN #2. FIGS. 1-3(a) show that, in the test formulations using OVA co-administered with the LNA formulations, anti-OVA-specific IgG levels were increased at both local and distant mucosal sites by several orders of magnitude relative to OVA co-administered with ODN, OVA co-administered with CT, OVA alone, or LNA-ODN #2. FIGS. 1 and 2(c) show that liposome encapsulation of the ODNs in the LNA formulations of the present invention increased anti-OVA IgM titers by several orders of magnitude relative to OVA co-administered with ODN, OVA co-administered with CT, OVA alone, or LNA-ODN #2. This response was dose dependent.

FIGS. 6 and 7 illustrate antibody titers in vaginal washes and lung washes. These figures show that, in the test formulations using OVA coadministered with the LNA formulations containing ODN #1, ODN #2 and ODN #3, the anti-OVA-specific IgA levels were increased at both local and distant mucosal sites by several orders of magnitude relative to OVA co-administered ODN #1, ODN#2 and ODN #3, LNA-ODN #2 alone, OVA co-administered with CT and PBS alone.

### Example 2

Stimulation of an Antigen-Specific Mucosal Immune Response Using OVA Coupled LNA formulations

This example illustrates the stimulation of a mucosal immune response to a target antigen using lipid-nucleic acid (“LNA”) formulations containing synthetic oligodeoxynucleotides having immunostimulatory CpG motifs (“ISS ODNS”) and co-administered with ovalbumin (“OVA”) as the target antigen.

Oligonucleotides

ISS ODN having 1 CpG motif, ODN #1 and ODN #2, were used in this Example and were synthesized with a phosphorothioate (“PS”) backbone (“ODN #1 PS” and “ODN #2 PS” respectively). The sequence of each ODN is provided above in Example 1.

Preparation of LNA Formulations

LNA formulations comprising ODN #1 PS or ODN #2 PS were prepared by formulating the ODNs with the lipid mixture DODAP:DSPC:CH:PEG-C14 (25:20:45:10 molar ratio), using the ethanol-based procedure fully described in U.S. Pat. Ser. No. 6,287,591 and incorporated herein by reference. Thereby, liposomes encapsulating ODN #1 (“LNA-ODN #1 PS”) or ODN #2 (“LNA-ODN #2 PS”) were prepared. Two different amounts of each ODN, 10 μg and 100 μg, were used to prepare the LNA formulations. The particle size of the resulting liposomes was 100-140 nm.

OVA coadminstered with CT was used as a control.

Preparation of OVA Coupled LNA Formuation

Two methods were used to prepare the formulation. Both methods rely on the OVA protein being activated by a thiolation procedure. The activated protein was chemically coupled directly to an active lipid species, for example DSPE-PEG-MPB with standard sulfahydryl chemistry (see, for example, Harasym et al., Bioconjugate Chemistry (1995) 6:187; Hermanson et al., Bioconjugate Techniques, Academic Press (1996) 230-232; Ansell et a., Antibody conjugation methods for active targeting of liposomes pages 51-68 in Drug targeting: strategies, principles and applications, Methods in Molecular Medicine, Vol 25, Edited by Francis, GE. and Delgado C., Human Press Inc., Totwa, New Jersey).

There are two ways of inserting the reactive lipid into the LNA formulation.

1) The reactive lipid is added when all the other lipid components of the LNA formuation are combined during the ethanol procedure described above. OVA is then coupled to the lipid after the lipid is in the formulation. This is called active coupling and is described in detail below.

2) The second method requires the lipid to first be combined with the OVA protein. This combined structure is then inserted into a preformed LNA formulation. This is called passive couples and again is described in detail below.

Thiolation of OVA Protein with SPDP

OVA (40 mg) dissolved in HBS (1 mol; 25 mM hepes, 150 mM NaCl, pH 7.4). A stock solution of SPDP (3-(2-pyridyidithio)propionic acid N-hydroxysuccinimide ester) was prepared in ethanol (28.8:1 EtOH/mg of SPDP) and an aliquot (40:1) added with vortexing to the OVA solution. The solution was stirred at room temperature for 30 minutes and passed down a Sephadex G-50 column (15 mil, SAS (sodium acetate saline) pH 4.4). Fractions were collected after 16 drops had fallen and analyzed at in a spectrophotometer at 280 nm, and fractions that were >1.0 at A₂₈₀ (absorbance at 280 nm) were combined. Typically 2.5-3 ml of protected thiolated protein is produced using this method. DTT (dithothreitol, 3.8 mg/ml solution) was then added directly as a solid and the solution stirred for 15 minutes. The solution was then passed down a sephadex G-50 column (50 ml in HBS, pH 7.4) collecting 20-24 drop fractions. The fractions were analyzed in a spectrophotometer at 280 nm, and those fractions that were >1.0 at A₂₈₀were combined. An aliquot of the combined fraction was then diluted 10 fold in HBS (Hepes Buffered Saline) and analyzed at 280 nm using HBS as a control sample. The protein content was determined by applying a factor of 1.8 to convert the absorbance into concentration (mg/ml).

Preparation of LNA-Protein Conjugates Using Active Coupling

Active coupling techniques refer to protocols in which an activated protein is chemically coupled directly to a reactive lipid incorporated into the lipid particle.

A solution of lipid comprised of DSPC/chol/MePEGS-2000-DMG/DODAP/DSPE-ATTA2-MPA (32:45:2:20:1 mol/mol) in ethanol (1.2 ml) was warmed to 60° C. and slowly added to a solution of ODN #2 (12 mg in 1.8 ml 300 mM citrate at PH 4.0), which had previously been warmed to 60° C. as well. The solution was vigorously agitated during addition. This crude LNA was then passed 10 times through two 100 nm filters using an extruder device set at 65° C. The resulting sized and crude LNA was then passed down a Sephadex G-50 column (50 ml; HBS) and used immediately. The approximate lipid concentration was estimated assuming that most of the lipid had been recovered from the column.

The thiolated OVA was then added to the activated LNA particles at an initial protein to lipid ratio of 150 g/mol and stirred at room temperature for 16 hours. The resulting LNA-protein (or LNA-OVA) conjugates were then separated from unreacted protein using Sepharose CI-4B columns (25 ml; HBS, ˜1 ml sample per column).

Preparation of LNA-Protein Conjugates Using Passive Coupling

Passive coupling techniques refer to protocols in which an activated protein is coupled to a reactive lipid remotely from the final lipid particle, and is then incorporated into the particle in some way, either by exchange into a preformed particle or by incorporation during the formation phase of the particle.

LNA particles were prepared as described above. A micellar solution of DSPE-ATTA2-MPA/DSPE-ATTA4-NBOC (1:4) was prepared by dissolving the lipid in a minimum of ethanol and slowly adding HBS, with a final lipid concentration at 10 mM. An aliquot of this solution was then added to the thiolated OVA described above in a ratio of 3000 g OVA/mol lipid and allowed to stir at room temperature overnight. An aliquot of this solution, corresponding to 150 g OVA/mol of lipid in the LNA, was added to a sample of the LNA and incubated in a water bath at 60° C. for an hour. This solution was then passed down a Sepharose CL-4B column (25 ml; HBS; ˜1 ml sample per column).

Immunization and Sample Isolation

C57BL/6 mice (6 weeks old) were immunized with 20 μl of the following test formulations by intranasal administration on day 0 (initial immunization), and days 7, and 14 after the initial immunization.

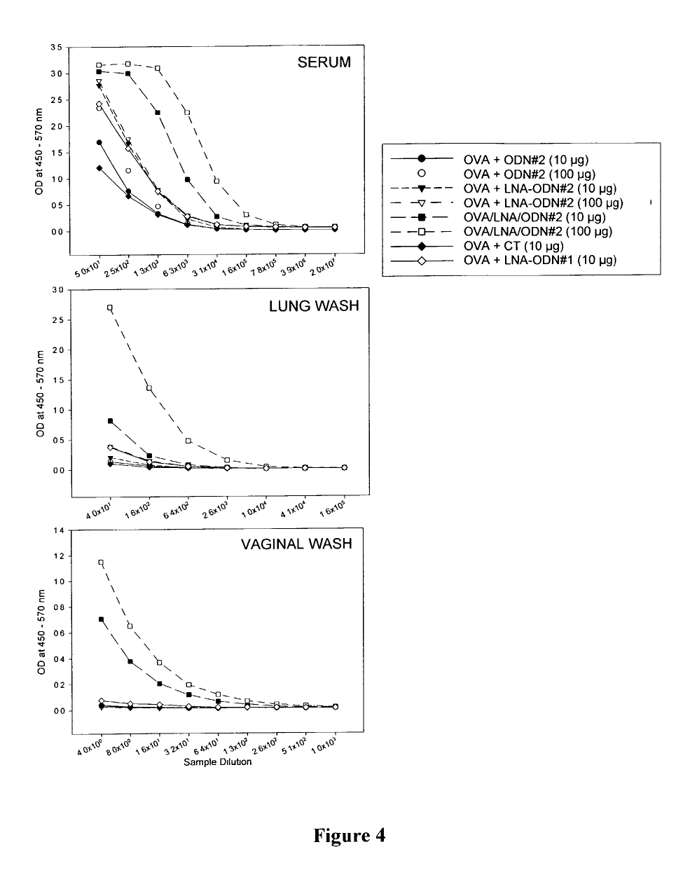

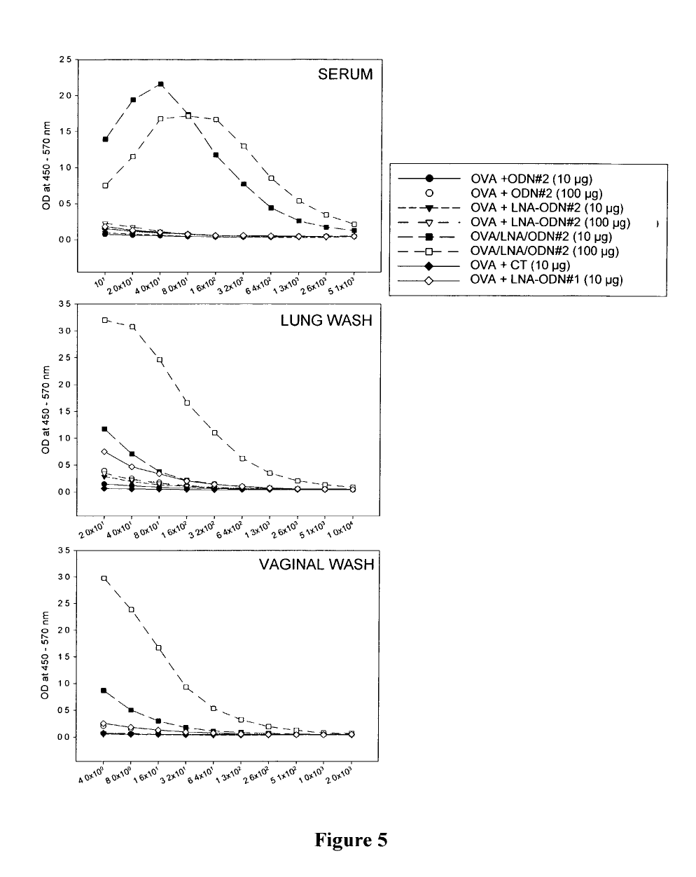

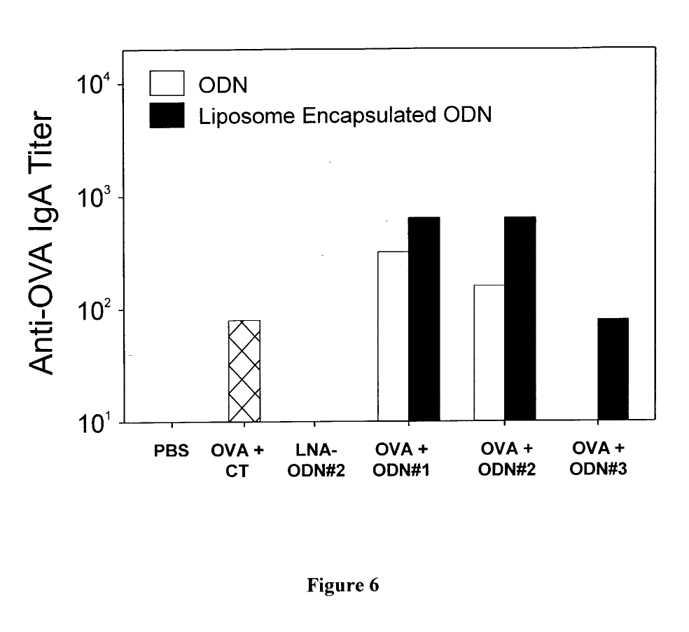

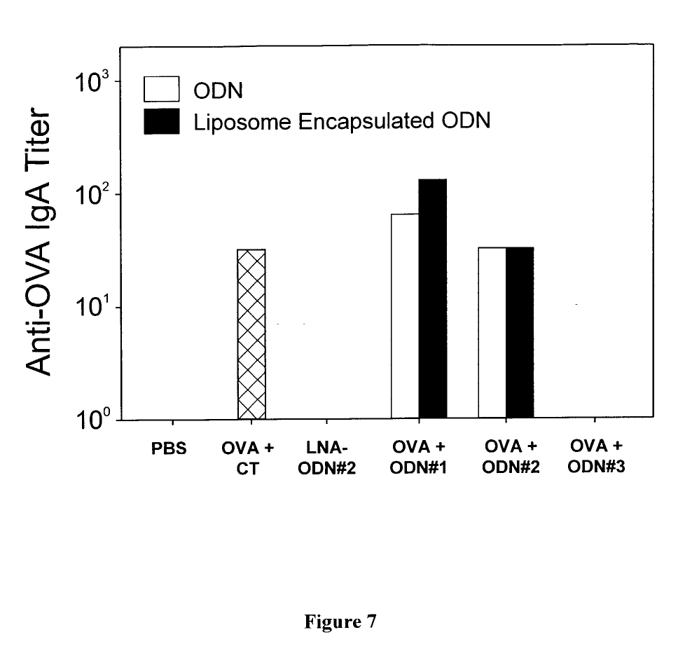

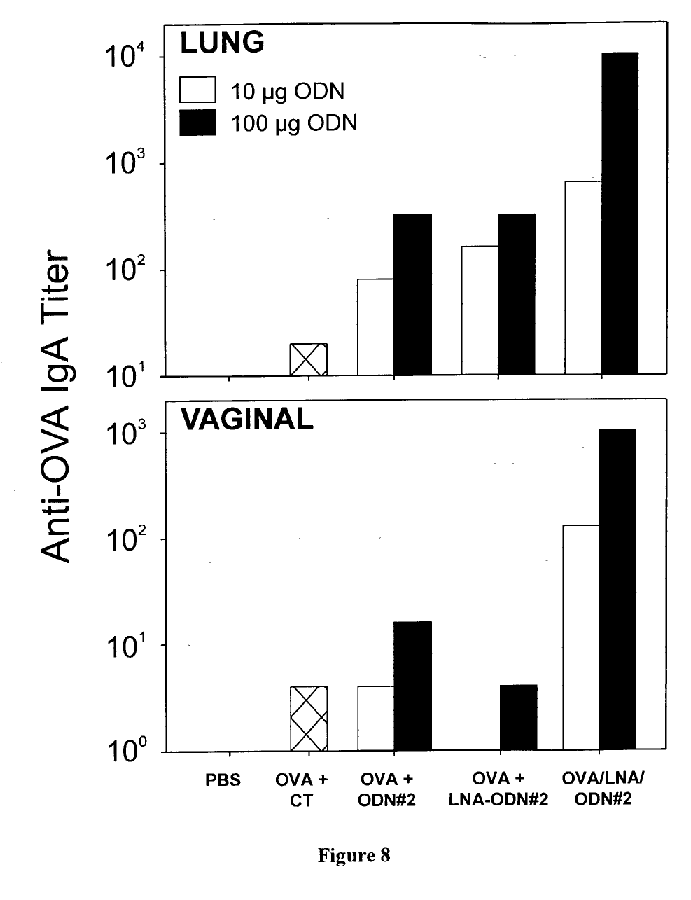

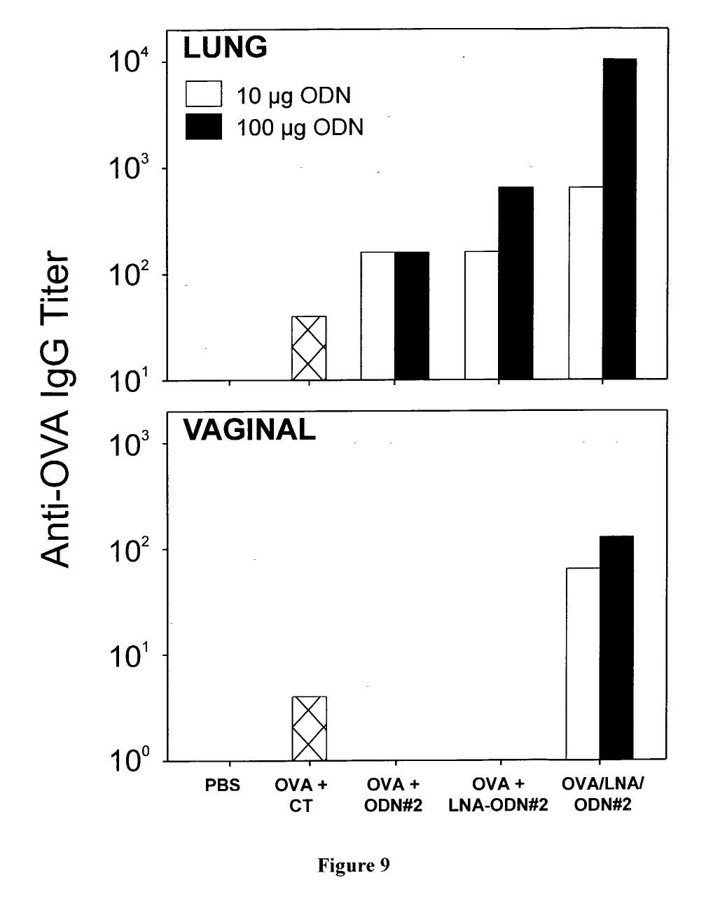

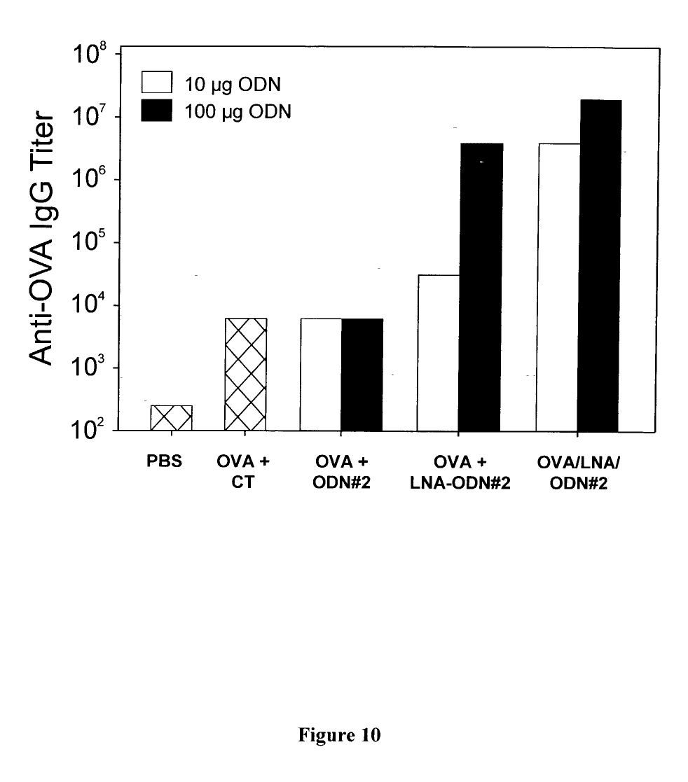

For FIGS. 4 and 5:

OVA co-administered with ODN #2 PS (“OVA+ODN #2 PS”) at a dose of 10μg

OVA co-administered with ODN #2 PS (“OVA+ODN #2 PS”) at a dose of 100 μg

OVA co-administered with LNA containing ODN #2 PS (“OVA+LNA-ODN #2 PS”) at a dose of 10 μg

OVA co-administered with LNA containing ODN #2 PS (“OVA+LNA #2 PS”) at a dose of 100 μg

OVA coupled to LNA containing ODN #2 (“OVA/LNA-ODN #2 PS”) at a dose of 10 μg

OVA coupled to LNA containing ODN #2 (“OVA/LNA-ODN #2 PS”) at a dose of 100 μg

OVA co-administered with 10 μg CT (“OVA+CT”)

OVA co-administered with LNA containing ODN #1 (“OVA/LNA-ODN #1 PS”) at a dose of 10 μg

Mice received OVA protein at a dose of 75 μg per immunization.

For FIGS. 8-10:

OVA coupled to LNA containing ODN #2 (“OVA/LNA-ODN #2 PS”) at a dose of 100 μg

OVA coupled to LNA containing ODN #2 (“OVA/LNA-ODN #2 PS”) at a dose of 10 μg

OVA co-administered with LNA containing ODN #2 PS (“OVA+LNA #2 PS”) at a dose of 100 μg

OVA co-administered with LNA containing ODN #2 PS (“OVA+LNA #2 PS”) at a dose of 10 μg

OVA co-administered with ODN #2 PS (“OVA+ODN #2 PS”) at a dose of 100 μg

OVA co-administered with ODN #2 PS (“OVA+ODN #2 PS”) at a dose of 10 μg

OVA co-administered with 10 μg of CT (“OVA+CT”) PBS alone

Mice received OVA protein at a dose of 75 μg per immunization.

Each treatment group (n=5) was anesthetized with halothane before droplets of the vaccine were applied to the external nares for complete inhalation. On day 28 after the initial immunization, plasma was collected from anesthetized mice by cardiac puncture and placed into serum tubes. Vaginal washes were obtained by pipetting 50 μl of PBS (Phosphate Buffer Saline) into and out of the vagina of each mouse. This procedure was repeated three times so that a total of 150 μl of washes were collected. The mice were subsequently terminated by cervical dislocation and a lung wash was performed by inserting tubing into the trachea and then pipetting 1 mil of PBS into and out of the lungs. Volume recovery for this precedure was generally 70-80%. Serum tubes were left at room temperature for 30 min to allow for clotting before centrifuging at 10,000 rpm (revolutions per minute) at 4° C. for 5 min, and the resulting aliquots of supernatent collected and stored at −20° C. until analysis. The serum aliquots were also stored at −20° C. until analysis.

ELISA Evaluation of the Immune Response

OVA-specific IgG and IgA antibodies in serum, lung washes, and vaginal washes were measured by ELISA (enzyme-linked immunosorbent assay). Microtiter plates (96 wells) were coated overnight at 4° C. with 5 μl/ml of OVA diluted in PBS (50 μl). The microtiter plates were then washed with PBS containing 0.5% Tween 20 (PBST) and blocked with 200 μl of 1% bovine serum albumin (BSA) in PBST for 1 hr at 37° C. with 50 μl of HRP (horse radish peroxidase)-conjugated goat anti-mouse IgG (1:4000) or HRP-conjugated sheep anti-mouse IgA (1:10) diluted with BSA-PBST. Plates were developed in a 30 min room temperature incubation with TMB. (100 μL) before stopping the reaction with 50 μl of 0.5 M H₂SO₃. Optical densities were read at 450-570 nm with an ELISA plate reader.

Results

Results are shown in FIGS. 4 and 5 illustrating antibody titers in serum, lung washes, and vaginal washes, FIGS. 8 and 9 illustrating antibody titers in vaginal washes and lung washes and FIG. 10 illustrating antibody titers in serum.

FIG. 5 shows that, in the test formulations using OVA coupled to the LNA formulations containing ODN #2, the anti-OVA-specific IgA levels were increased at both local and distant mucosal sites by several orders of magnitude relative to OVA co-administered with the LNA formulations containing ODN #2 or #1, OVA co-administered with ODN #2 and OVA co-administered with CT.

FIG. 4 shows that, in the test formulations using OVA coupled to the LNA formulations containing ODN #2, the, anti-OVA-specific IgG levels were increased at both local and distant mucosal sites by several orders of magnitude relative to OVA co-administered with the LNA formulations containing ODN #2 or #1, OVA co-administered with ODN #2 and OVA co-administered with CT.

FIG. 8 shows that, in the test formulations using OVA coupled to the LNA formulations containing ODN #2, the anti-OVA-specific IgA levels were increased at both local and distant mucosal sites by several orders of magnitude relative to OVA co-administered with the LNA formulations containing ODN #2, OVA co-administered with ODN #2, OVA co-administered with CT, PBS alone.

FIGS. 9 and 10 show that, in the test formulations using OVA coupled to the LNA formulations containing ODN #2, anti-OVA-specific IgG levels were increased at both local and distant mucosal sites by several orders of magnitude relative to OVA co-administered with the LNA formulations containing ODN #2, OVA co-administered with ODN #2, OVA co-administered with CT, or PBS alone.

In summary, this data demonstrates that IgA and IgG responses are greatly enhanced when the OVA is coupled to the LNA formulations. For example, mice immunized with OVA coupled to the LNA formulations containing ODN #2 of the present invention exhibited greater IgA titers in all fluids analyzed when compared to mice immunized with OVA mixed with free or encapsulated ODN #2 (see FIGS. 5 and 8). A dose response was also observed with the OVA coupled to the LNA formulations as a greater amount of ODN administered to the mice resulted in a larger production of IgA antibodies. This data demonstrate that coupling of OVA to LNA formulations can increase the ability of the LNA particles to generate IgA antibodies which has important implications for mucosal immunity. Further, mice immunized with OVA coupled to the LNA formulations containing ODN #2 of the present invention exhibited greater IgG titers in all fluids analyzed when compared to mice immunized with OVA mixed with free or encapsulated ODN #2 (see FIGS. 4, 9 and 10). A dose response was also observed with the OVA coupled to the LNA formulations as a greater amount of ODN administered to the mice resulted in a larger production of IgG antibodies. This data demonstrate that coupling of OVA to LNA formulations can increase the ability of the LNA particles to generate IgG antibodies.

We claim:

1. A method for stimulating an enhanced mucosal immune response in a mammal, said method comprising administering to said mammal an effective amount of an immunostimulatory composition comprising a lipid-nucleic acid (LNA) formulation associated with at least one antigen, wherein said LNA formulation comprises:
a) a lipid component comprising at least one lipid; and
b) a nucleic acid component comprising at least one oligonucleotide,
wherein said immunostimulatory composition stimulates an increased production of IgA as compared to the free form of said at least one oligonucleotide, in vivo.

2. The method according to claim 1, wherein said IgA production is at least two-fold greater as compared to the free form of said oligonucleotide mixed with said antigen.

3. The method according to claim 1, wherein said lipid component comprises a cationic lipid.

4. The method according to claim 3, wherein said cationic lipid is selected from a group of cationic lipids consisting of DDAB, DODAC, DOTAP, DMRIE, DOSPA, DMDMA, DC-Chol, DOGS, DODMA, and DODAP.

5. The method according to claim 3, wherein lipid component further comprises a neutral lipid selected from the group consisting of DOPE, DSPC, POPC, diacylphosphatidylcholine, diacylphosphatidylethanolamine, ceramide, sphingomyelin, cephalin, and cerebrosides.

6. The method according to claim 5, wherein said lipid component comprises DSPC, DODMA, Chol, and PEG-DMG and the ratio of said DSPC to said DODMA to said Chol to said PEG-DMG is about 20:25:45:10 mol/mol.

7. The method according to claim 6, wherein the ratio of said lipid component to said nucleic component is about 0.01-0.25 wt/wt.

8. The method according to claim 1, wherein said lipid component comprises a lipid membrane encapsulating said oligonucleotide.

9. The method according to claim 1, wherein said at least one oligonucleotide is an oligodeoxynucleotide (ODN).

10. The method according to claim 9, wherein said oligodeoxynucleotide (ODN) comprises at least one CpG dinucleotide.

11. The method according to claim 10, wherein said CpG dinucleotide is methylated or unmethylated.

12. The method according to claim 11, wherein said oligodeoxynucleotide (ODN) is selected from a group of ODNs consisting of ODN #1, ODN #2, ODN #3, ODN #4, ODN #5, ODN #6, ODN #7, ODN #8, and ODN #9.

13. The method according to claim 12, wherein said oligodeoxynucleotide (ODN) comprises a phosphorothioate backbone (ODN PS).

14. The method according to claim 1, wherein said lipid-nucleic acid (LNA) formulation further comprises an antigen.

15. The method according to claim 14, wherein said antigen is attached to said lipid-nucleic acid (LNA) formulation.

16. The method according to claim 15, wherein said lipid component comprises a lipid membrane having an external portion and an internal portion, and wherein said antigen is attached to said external portion of said lipid membrane.

17. The method according to any of claims 1-16, wherein said adminstering is by intranasal delivery.

18. The method according to any of claims 1-16, wherein said administering is by intradermal or subcutaneous delivery.

19. The method according to any of claim 1-16, wherein said administering is by ex vivo delivery.

20. An improved mucosal adjuvant comprising a lipid-nucleic acid (LNA) formulation, said LNA formulation comprising:
a) a lipid component comprising at least one lipid; and
b) a nucleic acid component comprising at least one oligonucleotide,
wherein said nucleic acid component is encapsulated by said lipid component, and said lipid component and said nucleic acid component act synergistically to stimulate immunoglobulin A (IgA) production in a mammal.

21. Use of the improved mucosal adjuvant according to claim 20 in combination with at least one antigen to stimulate antigen-specific IgA production in a mammal.

22. An improved mucosal vaccine composition comprising a lipid nucleic acid (LNA) formulation associated with at least one antigen, said LNA formulation comprising:
a) a lipid component comprising at least one lipid; and
b) a nucleic acid component comprising at least one oligonucleotide, wherein said nucleic acid component is encapsulated by said lipid component, and said lipid component and said nucleic acid component act synergistically to stimulate antigen-specific immunoglobulin A (IgA) production in a mammal.
